(19) United States
(12) Patent Application Publication
(10) Pub. No.: US 2025/0009872 A1
(43) Pub. Date: Jan. 9, 2025

(54) COMPOSITIONS AND METHODS FOR MODULAR VACCINES

(71) Applicant: Board of Regents of the University of Nebraska, Lincoln, NE (US)

(72) Inventors: Yuri Lyubchenko, Omaha, NE (US); Karen Zagorski, Omaha, NE (US); Mohtadin Hashemi, Omaha, NE (US)

(21) Appl. No.: 18/706,269

(22) PCT Filed: Dec. 14, 2022
(86) PCT No.: PCT/US22/81517

§ 371 Date: Apr. 30, 2024

Related U.S. Application Data
(60) Provisional application No. 63/289,279, filed on Dec. 14, 2021.

(51) Int. Cl. A61K 39/215; A61K 39/00; A61K 47/54; A61K 47/69; A61P 31/14; C07K 19/00; C12N 7/00

(52) U.S. Cl. CPC A61K 39/215; A61K 39/0011; A61K 47/549; A61K 47/6929; A61P 31/14; C07K 19/00; C12N 7/00; A61K 2039/55555; A61K 2039/6025; C12N 2770/20034

(57) ABSTRACT

Immunogenic constructs and vaccines are provided along with methods of use and manufacture thereof.

This application claims priority under 35 U.S.C. § 119 (e) to U.S. Provisional Patent Application No. 63/289,279, filed on Dec. 14, 2021. The foregoing application is incorporated by reference herein.

This invention was made with government support under Grant No. U54 GM115458 awarded by the National Institutes of Health. The government has certain rights in the invention.

### FIELD OF THE INVENTION

The present invention relates to the field of vaccines and the prevention of disease. More specifically the invention provides immunogenic constructs and methods of using such constructs for the inhibition, treatment, and/or prevention of disease.

### BACKGROUND OF THE INVENTION

The ongoing severe acute respiratory syndrome coronavirus 2 (SARS-COV-2) pandemic has demonstrated the need for vaccine technologies to ensure a rapid and efficient response to novel pathogens. Current vaccines for SARS-COV-2 are effective, but their inflammatory nature limits the use in individuals with immune disorders (Self, et al., Morb. Mortal. Wkly. Rep. (2021) 70 (38): 1337-43; Vuille-Lessard, et al., J. Autoimmun. (2021) 123:102710; Waqar, et al., Int. J. Hematol. (2021) 114 (5): 626-9; Velikova, et al., Rheumatol. Int. (2021) 41 (3): 509-18; Masset, et al., Kidney Int. (2021) 100 (2): 465-6; Karabulut, et al., Case Rep. Hematol. (2021) 2021:1-4; Diaz, et al., JAMA (2021) 326 (12): 1210). Moreover, herd immunity towards novel pathogens has proven unreliable and difficult to achieve, leaving immunocompromised individuals at risk (Thomson, H., New Sci. (2021) 251 (3348): 17; Aschwanden, et al., Nature (2021) 591 (7851): 520-2).

Immunocompromised individuals are at greater risk of infection and may remain infectious for longer than the immunocompetent, increasing the rate of community spread (Avanzato, et al., Cell (2020) 183 (7): 1901-12; Abbasi, J., JAMA (2021) 325 (20): 2033; Kaila, et al., Infect. Dis. (2021) 53 (11): 880-2). This highlights the need for vaccine platforms that can be individualized and adapted for immunocompromised individuals and individuals with autoimmune disorders (Rodríguez, et al., J. Autoimmun. (2020) 114:102506).

Conventional vaccines are composed of whole pathogens or their components. As a result, they can cause unwanted side effects, such as vaccine-enhanced infection, interfering neutralization antibodies, Hoskins effect, toxicity, and pathogen deattenuation (Vennema, et al., J. Virol. (1990) 64 (3): 1407-1409; Nicasio, et al., Viruses (2012) 4 (9): 1731-1752; Weingartl, et al., J. Virol. (2004) 78 (22): 12672-12676; Fox, et al., PLOS Pathog. (2016) 12 (1): 1005271; DeVries, et al., N. Engl. J. Med. (2011) 364 (24): 2316-2323). These issues put the immunocompromised vaccine recipients at even greater risk and further hinder vaccine development. Additionally, some current SARS-COV-2 vaccine recipients experience cardiovascular complications such as coagulopathies and myocarditis (Ostrowski, et al., Front. Immunol. (2021) 12:779453; Matta, et al., Cureus (2021) 13 (11):e19240).

Finally, vaccine development, production, and distribution are time-consuming and expensive, limiting their utility in fighting novel pathogens (Heaton, et al., Front. Immunol. (2020) 11:517290; Excler, et al., Nat. Med. (2021) 27 (4): 591-600). The currently approved vaccines for SARS-COV-2 are built upon prior developed SARS-COV and Middle East Respiratory Syndrome (MERS) vaccines. These seemingly quickly developed vaccines depended on prior knowledge about the immunopathological effects of targeting the nucleocapsid protein, as well as the 2p mutation used to stabilize the spike glycoprotein in the most immunogenic form (Yasui, et al., J. Immunol. (2008) 181 (9): 6337-6348; Kirchdoerfer, et al., Sci. Rep. (2018) 8 (1): 15701).

Based on the foregoing, it is clear that new vaccine strategies are required, particularly to protect against novel pathogens.

### SUMMARY OF THE INVENTION

In accordance with one aspect of the instant invention, immunogenic constructs are provided which can serve as vaccines. In certain embodiments, the immunogenic construct comprises a scaffold nucleic acid molecule and at least one complementary nucleic acid molecule, wherein the complementary nucleic acid molecule(s) is hybridized with the scaffold nucleic acid molecule, and wherein the complementary nucleic acid molecule(s) is conjugated to a peptide, particularly an antigen or immunogenic epitope. In certain embodiments, the immunogenic construct further comprises a complementary nucleic acid molecule conjugated to a T-helper cell epitope such as SEQ ID NO: 9. In certain embodiments, the immunogenic construct comprises a first complementary nucleic acid molecule conjugated to a first peptide antigen and a second complementary nucleic acid molecule conjugated to a second peptide antigen. In certain embodiments, the immunogenic construct comprises at least one peptide antigen to the S-protein of SARS-COV-2 (e.g., SEQ ID NO: 10 and/or SEQ ID NO: 11). In certain embodiments, the immunogenic construct comprises at least one tumor antigen. In certain embodiments, the immunogenic construct comprises at least one peptide antigen of amyloid β (e.g., SEQ ID NO: 8). In certain embodiments, the immunogenic construct further comprises a substrate for improved cellular uptake and/or biological stability conjugated to the scaffold nucleic acid molecule. In certain embodiments, the substrate is a nanoparticle, particularly a gold nanoparticle. In certain embodiments, the immunogenic construct further comprises a vaccine adjuvant.

Compositions comprising the immunogenic construct of the instant invention and at least one pharmaceutically acceptable carrier are also encompassed. In certain embodiments, the composition further comprises a vaccine adjuvant.

In accordance with another aspect of the instant invention, methods for inhibiting, treating, and/or preventing a disease in a subject in need thereof are provided. In certain embodiments, the method comprises administering an immunogenic construct of the instant invention to the subject. In certain embodiments, the disease is a viral infection, particularly a SARS-COV-2 infection. In certain embodiments, the disease is Alzheimer's disease. In certain embodiments, the disease is cancer.

### BRIEF DESCRIPTIONS OF THE DRAWING

FIG. 1A provides a schematic representation of an immunogenic construct of the invention. Three epitopes as peptides (P1-P3) are assembled on a DNA scaffold (with a thiol group connected by an adenosine linker (A9 (SEQ ID NO: 22)) containing thiol for coupling with gold. P1 (SEQ ID NO: 9) is the universal T helper cell epitope PADRE, P2 (SEQ ID NO: 10) is an S-protein-derived peptide (residues 1148-1159), which is important for cell membrane fusion, and P3 (SEQ ID NO: 11) is a peptide located near the receptor-binding domain (RBD) (residues 553-564), which is important for receptor recognition. FIG. 1B provides a graph of spectrophotometric melting observed by 260 nm absorbance change. The melting curve was taken at a temperature range from 10° C. to 85° C. FIG. 1C provides an image of agarose gel electrophoresis of immunogen gold formulations generated by different methods. From top to bottom: free gold nanoparticles, freezing-based conjugation of immunogen (FR), acid-based conjugation (PA), and salt-based conjugation (SC). FIG. 1D provides an AFM image of a dry sample of gold nanoparticles in air. The image size is 3 μm×3 μm, and the Z-scale is 16 nm. FIG. 1E provides a histogram of the values of the diameters of the particles fitted with a Gaussian curve. The mean value is 35.4+/−0.1 nm (SEM).

FIG. 2A provides a graph showing a comparison between conjugation methods characterized by DLS, AFM and Zeta potential. Error bars are shown in all histograms. FIG. 2B provides a graph of the cytotoxicity of the four formulations in Vero-E6 cells with or without adjuvant measured using a standard colorimetric MTT assay that measures cell proliferation and viability. The percent viability of the cells is plotted. The freezing (FR), salt-based (SC), pH-assisted (PA) and gold-free inulin (IN) based conjugated immunogen show no toxicity in Vero-E6 cells. Further, when combined with the CpG adjuvant (Adj), zero toxicity in Vero-E6 cells was found. These experiments were performed twice, representing the mean SEM from three replicates.

FIGS. 3A-3C show the IgG immune response against P1 and P2 in mice immunized with GNP-conjugated vaccine candidates. FIG. 3A provides a schematic of the experiments. BALB/c mice were immunized with GNP-conjugated vaccine candidates at different time intervals (Day 0, 14, 28, and 61), and blood and spleen were collected as shown. High binding 96 well plates were separately coated with P1 and P2 (100 ng/well) overnight at 4° C. Sera prepared from different timepoints of immunization were diluted at 1:25 and incubated for 1 hour at room temperature. IgG immune response against P1 (FIG. 3B) and P2 (FIG. 3C) was determined using HRP-conjugated goat anti-mouse IgG in the presence of TMB as substrate. * P<0.05.

FIGS. 4A and 4B show the IgG immune response against SARS-COV-2 whole spike and RBD in mice immunized with GNP-conjugated vaccine candidates. High binding 96 well plates were separately coated with whole spike or RBD (1 μg/ml) overnight at 4° C. Sera prepared from different time immunization points were diluted at 1:25 and incubated for 1 hour at room temperature. IgG immune response against the spike (FIG. 4A) and the RBD (FIG. 4B) was determined. * P<0.05.

FIGS. 5A-5C show the SARS-COV-2 live virus neutralization ability of sera from mice immunized with GNP-conjugated vaccine candidates. Sera (Day61) from all groups of immunized mice were evaluated for live virus neutralization ability against SARS-COV-2 isolate USA-WA1/2020 (WT), SARS-COV-2 isolate USA-WI1/2020 (WI), and SARS-COV-2 isolate hCoV-19/USA/PHC658/2021 (delta). Sera (5×diluted, starting from 1:25) were separately incubated with WT, WI, and delta viruses at 10⁴ PFU for 1 hour. Next, the virus-sera mix was added to the cells and incubated for 24 hours. Cells were then fixed and permeabilized and stained with the combination of anti-SARS-COV-2 spike protein antibodies (rabbit mAb) and Alexa Fluor® 488 goat anti-rabbit as primary and secondary antibodies, respectively. The nuclei were stained using Hoechst 33342 and plates were read using Operetta Imager. The percent neutralization was calculated based on the differential intensity of the fluorescence.

FIG. 6 provides a SARS-COV-2 VOCs map of mutations spanning from RBD to heptad repeat 2 (HR2). The four spike protein variants: beta, gamma, delta, and omicron BA1, are represented schematically. The N-terminal domain and mutations localized within are omitted. Selected epitopes are expected to disrupt the activity of the subdomain 1 (SD1) and heptad repeat 2 (HR2), thus blocking viral attachment and cellular entry. The information about mutations is taken from the covdb.stanford.edu database.

FIG. 7A provides a schematic of the assembly of a peptide dimer by the annealing of complementary DNA strands. Peptides are tethered to the DNA strand via a flexible polymer. FIG. 7B provides a schematic of the assembly of a DNA-Aβ (14-23) dimer. (S18)₃ phosphoramidite flexible tethers may be synthesized along with the DNA. S18: Spacer 18 phosphoramidite (18-O Dimethoxytritylhexaethyleneglycol, 1-[(2-cyanoethyl)-(N,N-diisopropyl)]-phosphoramidite. Peptides may be conjugated to the active ends of the tether (DBCO residues) via click chemistry. Top strand is SEQ ID NO: 12 and bottom strand is SEQ ID NO: 13. FIG. 7C provides a schematic of an example of a dimer vaccine. A long DNA template is provided to accommodate the peptide dimer and DNA conjugated with a T helper cell epitope PADRE peptide (Pan DR-biding epitope). FIG. 7D provides a schematic of an example of a dimer vaccine. Two short DNA oligonucleotides organized in series and ligated provide the dimer assembly of the peptides. The third DNA strand contains a PADRE peptide.

FIG. 8 provides a schematic of the assembly of an amyloid beta dimer tethered to a hybrid flexible polymeric tether.

FIG. 9 provides a schematic of the assembly of an amyloid beta tetramer tethered to a flexible polymeric tether.

FIG. 10 provides a schematic of the assembly of an amyloid beta octamer (left) and hexamer (right) tethered to a flexible polymeric tether.

FIG. 11 provides a schematic of an immunogen for a tetramer with the additional T helper cell epitopes to enhance immune response formulated with the nanoparticle. The top strand DNA-2 is used for the ligation of the tetramer assembly to the epitope assembly. A thiol-gold bond is used to conjugate the immunogen to the nanoparticle shown to the right.

### DETAILED DESCRIPTION OF THE INVENTION

Herein, a novel approach in vaccinology is provided, including a proof-of-concept modular epitope vaccine against SARS-COV-2. The strategy is based on short peptide epitopes that generate immune responses precisely aimed at the critical components of the SARS-COV-2, limiting the risk of adverse effects (Wisnewski, et al., PLOS ONE (2021) 16 (9): 0252849; Palatnik-de-Sousa, et al., Front. Immunol. (2018) 9:826; Chauhan, et al., Sci. Rep. (2019) 9 (1): 2517). The immunogen is assembled on a long single-stranded DNA scaffold to which short DNA probes are annealed in a sequence-specific manner. Each DNA probe carries a specific epitope covalently attached to the end of the probe. Two peptides corresponding to the well-identified linear epitopes from the S-protein of SARS-COV-2 were used (Li, et al., Cell. Mol. Immunol. (2020) 17 (10): 1095-1097). The Pan HLA DR-binding Epitope (PADRE) (Rosa, D., Immunol. Lett. (2004) 92 (3): 259-268) was introduced via attachment to one of the probes to endow the vaccine with antigenicity. Each probe-epitope conjugate and the scaffold DNA are prepared individually and can be modified or replaced, making the immunogen truly modular. Several formulations of the immunogen were prepared, and mice were immunized to evaluate the spike- and peptide-specific immune responses. The novel vaccine design generated immune responses in mice without any adverse events, and the serum from these mice were able to neutralize the wild type and the Delta variant of SARS-COV-2.

The current vaccine development strategies for the COVID-19 pandemic utilize whole inactive or attenuated viruses, virus-like particles, recombinant proteins, and antigen-coding DNA and mRNA with various delivery strategies. While highly effective, these vaccine development strategies are time-consuming and often do not provide reliable protection for immunocompromised individuals, young children, and pregnant women. Here, a novel modular vaccine platform is provided which addresses these short comings using chemically synthesized peptides identified based on the validated bioinformatic data about the target. The vaccine is based on the rational design of an immunogen containing two defined B-cell epitopes from the spike glycoprotein of SARS-COV-2 and the universal T-helper epitope PADRE. The epitopes were conjugated to short DNA probes and combined with a complementary scaffold strand, resulting in sequence-specific self-assembly. The immunogens were then formulated by conjugation to gold nanoparticles by three methods or by co-crystallization with epsilon insulin. BALB/C mice were immunized with each formulation, and the IgG immune responses and virus neutralizing titers were compared. The results demonstrate that this assembly is immunogenic and generates specific neutralizing antibodies against wild-type SARS-COV-2 and the Delta variant. Notably, the peptides used do not belong to the RBD segment of the S-protein and do not contain mutations in SARS-COV-2 variants of concern or interest, including Delta and Omicron variants, thereby showing the ability of the immunogenic construct to induce antibodies against a wide array of SARS-COV-2 variants. The use of the non-toxic DNA scaffold as a platform allows for the assembly of the immunogen as one unit with reproducible and predictable assembly, allowing for incorporation of the above components as well as additional peptide epitopes and/or immunostimulants.

The modular feature of the invention provides numerous advantages over existing vaccines. For example, the invention allows for a rapid response to an infection and accelerates the development of new vaccines. Additionally, it addresses the major issues related to traditional vaccine development approaches associated with side effects, failures to induce protection, or even worsening of the infection through antibody enhanced infection or neutralization interfering antibodies. The modular feature of the invention also significantly reduces the amount of resources required for production. Moreover, the use of short peptides (epitopes) instead of large proteins dramatically decreases the cost of the vaccine assembly. Further, the use of short peptides instead of large, engineered proteins avoids unwanted side effects such as different patterns of proteolytic processing and production of new, uncharacterized and unwanted epitopes. The superior response times and manufacturing costs make the vaccine accessible to larger populations in times of need, before they get infected. Further, the vaccine of the instant invention has excellent stability and vaccine storage does not require low temperatures. Indeed, the vaccine can be stored in a refrigerator. This invention improves healthcare access and improves the capacity to contain infections.

In accordance with the present invention, constructs, particularly immunogenic constructs, are provided. In certain embodiments, the immunogenic constructs are vaccines. The immunogenic constructs of the instant invention can, upon administration to a host, induce an immunogenic response (e.g., a humoral response and/or active immune response) against a target protein(s).

The immunogenic constructs or vaccines of the present invention can be used to inhibit, treat, and/or prevent a disease or disorder in a subject. In certain embodiments, the disease or disorder is an infectious disease. In certain embodiments, the disease or disorder is cancer. In certain embodiments, the disease or disorder is a viral infection. Examples of viral infections include, but are not limited to, infections by: influenza A, influenza B, influenza H1N1, avian influenza (e.g., H5N1), SARS-COV, SARS-COV-2, MERS, Chikungunya, flavivirus (tick encephalitis), Zika, Dengue, Marburg, Ebola, Hepatitis C virus, and Andes hantavirus. In certain embodiments, the disease or disorder is SARS-COV-2. In certain embodiments, the disease or disorder is characterized by a pathogenic protein. In certain embodiments, the disease or disorder is Alzheimer's disease.

In certain embodiments, the immunogenic construct of the instant invention comprises a scaffold nucleic acid molecule or oligonucleotide and one or more complementary nucleic acid molecules or oligonucleotides linked or conjugated to at least one peptide, particularly a peptide epitope (e.g., immunogen or antigen), particularly a disease specific peptide. In certain embodiments, the scaffold nucleic acid molecule comprises DNA. In certain embodiments, the scaffold nucleic acid molecule is single-stranded. In certain embodiments, the scaffold nucleic acid molecule is linked or conjugated to at least one peptide, particularly a peptide epitope (e.g., immunogen or antigen), particularly a disease specific peptide. In certain embodiments, the complementary nucleic acid molecule comprises DNA. In certain embodiments, the complementary nucleic acid molecule is single stranded.

The immunogenic construct may comprise any number of peptides and/or complementary nucleic acid molecules linked or conjugated to a peptide. In certain embodiments, the immunogenic construct comprises 1-50 peptides or peptide antigens and/or complementary nucleic acid molecules linked or conjugated to a peptide. In certain embodiments, the immunogenic construct comprises 1-10 peptides or peptide antigens and/or complementary nucleic acid molecules linked or conjugated to a peptide. In certain embodiments, the immunogenic construct comprises 1˜4 peptides or peptide antigens and/or complementary nucleic acid molecules linked or conjugated to a peptide. In certain embodiments, the immunogenic construct comprises at least two, at least three, or at least four complementary nucleic acid molecules. When an immunogenic construct comprises more than one complementary nucleic acid molecule, each of the complementary nucleic acid molecules may be conjugated (e.g., via a linker) to a unique peptide (i.e., the peptide conjugated to a first complementary nucleic acid molecule is different than the peptide conjugated to a second complementary nucleic acid molecule). In certain embodiments, all of the peptides of the immunogenic construct are from one disease or disorder (e.g., all of the peptide antigens are from one virus or viral genus). In certain embodiments, the peptides of the immunogenic construct are from more than one disease or disorder (e.g., the peptide antigens are from more than one virus or viral genus), thereby allowing the targeting of more than one disease.

Generally, the sequence of the complementary nucleic acid molecules is completely complementary to the sequence of the scaffold nucleic acid molecule. While one or more mismatches can be tolerated, it is desirable to have complete complementarity for more stable binding of the complementary nucleic acid molecules to the scaffold nucleic acid molecule. Typically, the length of the scaffold nucleic acid molecule will be only as long as needed to bind the complementary nucleic acid molecules. The binding regions for the complementary nucleic acid molecules can be immediately adjacent to each other or have a gap of nucleotides (e.g., 1-3 nucleotides, 1-5 nucleotides, 1-8 nucleotides, 1-10 nucleotides, or 1-15 nucleotides) or other distance between the binding regions. In certain embodiments, there are no nucleotides between the binding regions for the complementary nucleic acid molecules. As an example, if the scaffold nucleic acid comprises the sequence:

(SEQ ID NO: 14)
CCTTCATCCTTACTGAGCTAGTCGAACGTCAGGAA
CGTCATGGACCTTGCGACAGACAGCCTACTCAC,

then complementary nucleic acid molecules may comprise sequences such as, without limitation: TAAGGATGAAGG (SEQ ID NO: 15), CGACTAGCTCAG (SEQ ID NO: 16), TCCATGACGTTCCTGACGTT (SEQ ID NO: 17), TCTGTCGCAAGG (SEQ ID NO: 18), and GTGAGTAGGCTG (SEQ ID NO: 19). Notably, there are no gaps or nucleotides between the binding regions for the complementary nucleic acid molecules in the scaffold nucleic acid in this example.

As stated hereinabove, the scaffold nucleic acid molecule will generally have a length that is sufficient to bind the desired number of complementary nucleic acid molecules. In certain embodiments, the scaffold nucleic acid molecule is less than 500 nucleotides (or equivalents thereof) in length, less than 400 nucleotides (or equivalents thereof) in length, less than 300 nucleotides (or equivalents thereof) in length, less than 300 nucleotides (or equivalents thereof) in length, less than 100 nucleotides (or equivalents thereof) in length, less than 75 nucleotides (or equivalents thereof) in length, or less than 50 nucleotides (or equivalents thereof) in length. In certain embodiments, the scaffold nucleic acid molecule comprises SEQ ID NO: 14. In certain embodiments, the scaffold nucleic acid molecule comprises TATAGTGAGTAGGCTGTATA-TATACGACTAGCTCAGTATA-TATATAAGGATGAAGGTATA (SEQ ID NO: 23).

In certain embodiments, the scaffold nucleic acid molecule is functionalized (e.g., at the 3′ or 5′ end) to allow conjugation to a substrate such as a nanoparticle. For example, the scaffold nucleic acid molecule may comprise a thiol (—SH) group (e.g., at the 3′ or 5′ end). The functional group (e.g., thiol group) may be conjugated to the scaffold nucleic acid molecule via a linker with a terminal thiol group. In certain embodiments, the linker is a nucleic acid molecule (e.g., DNA (e.g., an adenosine stretch)), particularly a nucleic acid molecule of 1-25 nucleotides, 1-20 nucleotides, 1-15 nucleotides, 5-20 nucleotides, or 5-15 nucleotides in length.

Generally, the complementary nucleic acid molecules will be only of sufficient length to ensure stable hybridization with the scaffold nucleic acid molecule. In certain embodiments, the complementary nucleic acid molecules will be only of sufficient length to ensure stable hybridization, at minimum at body temperature, with the scaffold nucleic acid molecule In certain embodiments, the complementary nucleic acid molecules are less than about 50 nucleotides, less than about 40 nucleotides, less than about 30 nucleotides, less than about 25 nucleotides, less than about 20 nucleotides, or less than about 15 nucleotides in length. In certain embodiments, the complementary nucleic acid molecules are greater than about 8 nucleotides, greater than about 9 nucleotides, greater than about 10 nucleotides, greater than about 11 nucleotides, or greater than about 12 nucleotides in length. In certain embodiments, the complementary nucleic acid molecule is about 10 to about 30 nucleotides in length, about 10 to about 25 nucleotides in length, about 15 to about 30 nucleotides in length, or about 15 to about 25 nucleotides in length. In certain embodiments, the complementary nucleic acid molecule comprises SEQ ID NO: 4. In certain embodiments, the complementary nucleic acid molecule comprises SEQ ID NO: 5. In certain embodiments, the complementary nucleic acid molecule comprises SEQ ID NO: 6. In certain embodiments, the complementary nucleic acid molecule comprises SEQ ID NO: 15. In certain embodiments, the complementary nucleic acid molecule comprises SEQ ID NO: 16. In certain embodiments, the complementary nucleic acid molecule comprises SEQ ID NO: 17. In certain embodiments, the complementary nucleic acid molecule comprises SEQ ID NO: 18. In certain embodiments, the complementary nucleic acid molecule comprises SEQ ID NO: 19.

Peptide antigens or epitopes are known in the art for many of the causative agents of diseases and disorders. In certain embodiments, the peptide antigen or epitope is neutralizing antibody inducing. In certain embodiments, the peptides are immunogens (e.g., viral immunogen sequences) such as primary B-cell epitopes and/or cytotoxic (CD8+) T-cell epitopes. For new or unknown pathogens, computational bioinformatic methods may be used to determine potential epitopes based on surface exposure of sequences and on homology with known epitopes. Also, phage display library as well as alanine scanning techniques can be used to determine new epitopes based on existing neutralizing antibodies from patients. In certain embodiments, the peptide conjugated to the complementary nucleic acid is a fragment and/or linear peptide (e.g., consecutive amino acids) from the target protein (e.g., a surface protein (e.g., spike protein) of a virus, tumor antigen (e.g., surface protein of tumor or cancer), or amyloid β for Alzheimer's disease). In certain embodiments, the peptide of the instant invention is fewer than 50 amino acids, fewer than 40 amino acids, fewer than 35 amino acids, fewer than 30 amino acids, fewer than 25 amino acids, or fewer than 20 amin acids in length. In certain embodiments, the peptide of the instant invention is greater than 5 amino acids, greater than 6 amino acids, greater than 7 amino acids, greater than 8 amino acids, greater than 9 amino acids, or greater than 10 amino acids in length. In certain embodiments, the peptide is about 5 to about 50 amino acids, about 5 to about 40 amino acids, about 5 to about 35 amino acids, about 8 to about 30 amino acids, about 8 to about 20 amino acids, about 8 to about 19 amino acids, about 8 to about 18 amino acids, about 9 to about 18 amino acids, about 8 to about 17 amino acids, about 9 to about 16 amino acids, about 10 to about 16 amino acids, or about 10 to about 15 amino acids in length.

In certain embodiments, the peptide is an epitope from the spike protein of SARS-COV. Examples of neutralizing antibody inducing epitopes for SARS-COV include, without limitation: FSPDGKPCTPPALNCYWPLNDYGFYTTTGIGYQ (SEQ ID NO: 20) and FSPDGKPATPPALNAYW (SEQ ID NO: 21) (Berry, et al., mAbs (2010) 2:53-66, incorporated by reference herein).

In certain embodiments, the peptide is an epitope from the spike protein of SARS-COV-2. Examples of neutralizing antibody inducing epitopes for SARS-COV-2 include, without limitation: SEQ ID NO: 10 and SEQ ID NO: 11.

In certain embodiments, the peptide is an epitope from amyloid β. In certain embodiments, the peptide epitope from amyloid β comprises Aβ (14-23) or SEQ ID NO: 8.

The amino acid sequence of the peptides of the instant invention may have at least 75%, at least 80%, at least 85%, at least 90%, at least 95%, at least 97%, at least 99%, or 100% homology or identity with any peptide described herein (e.g., SEQ ID NOs: 8, 9, 10, 11, 20, or 21), particularly at least 90% homology or identity (e.g., the sequence may contain additions, deletions, and/or substitutions). In certain embodiments, the peptide of the instant invention may extend beyond any peptide described herein (e.g., SEQ ID NOs: 8, 9, 10, 11, 20, or 21) at the amino and/or carboxyl terminus by 1, 2, 3, 4, 5, 6, 7, 8, 9, or 10 amino acids, particularly by 1, 2, 3, 4, or 5 amino acids, by 1, 2, or 3 amino acids, by 1 or 2 amino acids, or by 1 amino acid of the sequence in the target protein.

The peptide of the instant invention may have capping, protecting and/or stabilizing moieties at the C-terminus and/or N-terminus (e.g., the termini opposite the one conjugated to the nucleic acid molecule). Such moieties are well known in the art and include, without limitation, amidation and acetylation. In a particular embodiment, the peptides of the instant invention are amidated (e.g., at the C-terminus).

The complementary nucleic acid molecules can be conjugated to the peptides either directly or via a linker using methods known in the art. In certain embodiments, the complementary nucleic acid molecules are conjugated to the C-terminus of the peptide. In certain embodiments, the complementary nucleic acid molecules are conjugated to the N-terminus of the peptide. In certain embodiments, the linker is a nucleic acid molecule.

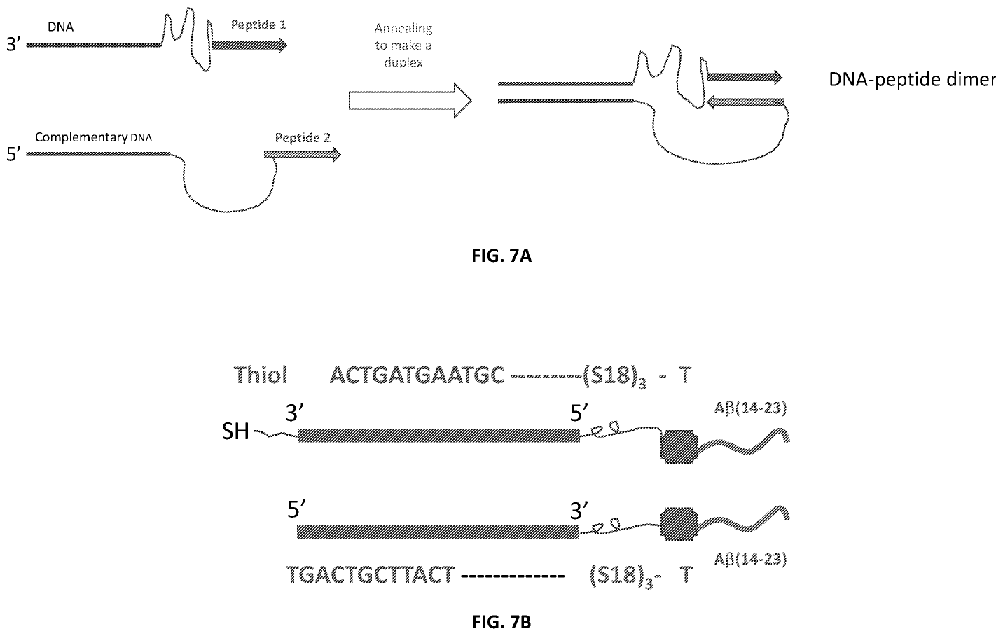

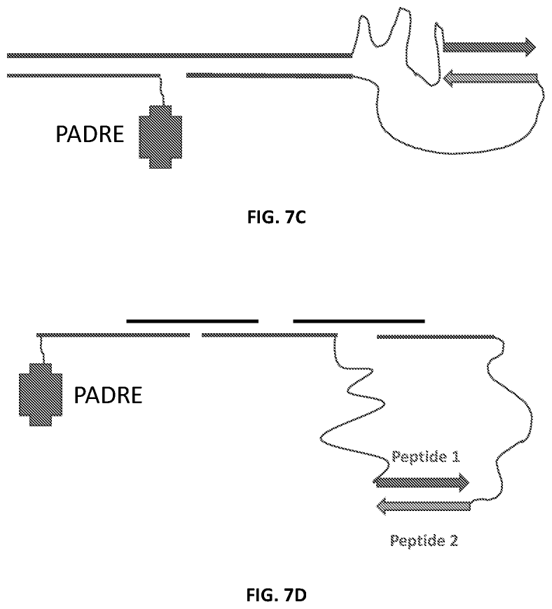

In certain embodiments, the peptide epitope forms a multimeric complex (e.g., dimer, trimer, tetramer, or octamer). In certain embodiments, the peptide that forms a multimeric complex is an amyloid β peptide. In certain embodiments, to facilitate the formation of the multimeric complex, the peptide can be attached to the scaffold nucleic acid molecule and/or complementary nucleic acid molecule via a flexible linker. Examples of such arrangements are depicted, for example, in FIGS. 7-11.

The term “linker” refers to a chemical moiety comprising a covalent bond or a chain of atoms that covalently attach at least two compounds. The linker can be linked to any synthetically feasible position of the compounds, but preferably in such a manner as to avoid blocking the compound's desired activity. Linkers are generally known in the art. In certain embodiments, the linker may contain from 0 (i.e., a bond) to about 100 atoms, from 0 to about 50 atoms, from 0 to about 25 atoms, from 0 to about 10 atoms, or from about 1 to about 5 atoms. In certain embodiments, the linker comprises nucleic acids and/or analogs thereof.

Exemplary linkers may comprise at least one optionally substituted; saturated or unsaturated; linear, branched or cyclic aliphatic group, an alkyl group, or an optionally substituted aryl group. Specific examples of linkers that can be used to link the complementary nucleic acid molecule to the peptide include but are not limited to PolyT and/or Spacer Phosphoramidite 18 (18-O-Dimethoxytritylhexaethyleneglycol, 1-[(2-cyanoethyl)-(N,N-diisopropyl)]-phosphoramidite). These linkers provide additional flexibility of attachment site. Examples of other flexible linkers include, without limitation: phosphoramidites with more or less than 6 units of ethylene glycol or hydrophobic spacers (Spacer Phosphoramidite 9; Spacer C12 CE Phosphoramidite) or abasic sites (dSpacer CE Phosphoramidite).

In certain embodiments, the complementary nucleic acid molecule is conjugated to the peptide by click chemistry. In certain embodiments, the peptide is conjugated to a flexible linker by click chemistry. In certain embodiments, the click chemistry reaction is an azide-alkyne cycloaddition (e.g., Pickens et al., Bioconjugate Chem. (2018) 29 (3): 686-701). In certain embodiments, the peptide is functionalized with an azide group (N₃) or a compound comprising an azide group (e.g., azidolysine). In certain embodiments, the complementary nucleic acid molecule or flexible linker is functionalized with an alkyne group (carbon-carbon triple bond). In certain embodiments, the reactant of the click chemistry reaction is covalently attached to the peptide directly or via a linker. In certain embodiments, a compound comprising the reactant of the click chemistry reaction is covalently attached to the complementary nucleic acid molecule directly or via a linker.

Examples of compounds comprising alkyne include, without limitation: DBCO, DIBAC, DIFO, DIBO, BCN, BARAC, and derivatives thereof. In certain embodiments, the complementary nucleic acid molecule is covalently attached directly or via a linker to a compound comprising dibenzocyclooctyne (DBCO). Other alkynes for bioorthogonal click chemistry, include, without limitation: DBCO-dT-CE Phosphoramidite (5′-Dimethoxytrityl-5-[(6-oxo-6-(dibenzo[b,f]azacyclooct-4-yn-1-yl)-capramido-N-hex-6-yl)-3-acrylimido]-2′-deoxyUridine,3′-[(2-cyanoethyl)-(N,N-diisopropyl)]-phosphoramidite) and C8-Alkyne-dT-CE Phosphoramidite (5′-Dimethoxytrityl-5-(octa-1,7-diynyl)-2′-deoxyuridine, 3′-[(2-cyanoethyl)-(N,N-diisopropyl)]-phosphoramidite). Other alkyne reagents include, without limitation: various cyclic and terminal alkynes as well as silyl protected terminal alkynes (e.g., 5′-Dimethoxytrityl-5-[8-trimethylsilyl-octa-1,7-diynyl]-2′-deoxyuridine,3′-[(2-cyanoethyl)-(N,N-diisopropyl)]-phosphoramidite). Alternatively halogenated phosphoramidites (such as 5′-Iodo-2′-deoxyThymidine,3′-[(2-cyanoethyl)-(N,N-diisopropyl)]-phosphoramidite) can be used for quantitative conversion into organic azides. Also, diels-alder protected maleimide (2-(1,7-Dimethyl-3,5-dioxo-10-oxa-4-azatricyclo[5.2.1.02,6]dec-8-en-4-yl)-ethyl-1-O-[(2-cyanoethyl)-(N,N-diisopropyl)]-phosphoramidite) can be incorporated for thiol-Michael addition click chemistry with cysteines.

In certain embodiments, the immunogenic construct comprises a complementary nucleic acid molecule conjugated to a T-cell epitope such as a cytotoxic T-cell epitope or a T-helper cell epitope. In certain embodiments, the immunogenic construct comprises a scaffold nucleic acid molecule conjugated to a T-cell epitope. The T-cell epitope may be a peptide and conjugated (or linked) to the nucleic acid molecule (e.g., complementary nucleic acid molecule) as described herein for other peptides such as immunogenic peptides. In certain embodiments, the complementary nucleic acid molecule is conjugated to a T-helper cell epitope. The T-helper cell epitopes can be used to stimulate T helper mediated initiation and activation of the immune response. Examples of T helper cell epitopes include, without limitation: PADRE peptides (e.g., Alexander, et al., Immunity (1994) 1:751-761, incorporated by reference herein), HIV-1 reverse transcriptase derived RT171-190 peptide (e.g., van der Burg, et al., J. Immunology (1998) 162:152-160, incorporated by reference herein), HIV-1 derived peptides (e.g., Pol 303, Pol 335, Pol 711, Gag 171; e.g., Livingston, et al., J. Immunology (2002) 168:5499-5506, incorporated by reference herein), T* peptides from Plasmodium falciparum (e.g., Parra-Lopez, et al., J. Biol. Chem. (2006) 281:14907-14917, incorporated by reference herein), CD4 Helper memory TpD peptides containing epitopes to tetanus and diphtheria toxoid (e.g. Fraser, et al., Vaccine (2014) 32:2896-2903, incorporated by reference herein), diphtheria toxoids (DTDs) and tetanus toxoids (TTDs) peptides/epitopes (e.g., Diethelm-Okita, et al., J. Infectious Diseases (2000) 181:1001-1009, incorporated by reference herein), and tetanus toxoid derived epitopes (e.g., Panina-Bordignon, et al., Eur. J. Immunology (1989) 19:2237-2242, incorporated by reference herein). In certain embodiments, the T-helper cell epitope is a PADRE peptide. In certain embodiments, the T-helper cell epitope comprises GSAKFVAAWTLKAAA (SEQ ID NO: 9).

In certain embodiments, the immunogenic construct is also conjugated to a vaccine adjuvant. In certain embodiments, the vaccine adjuvant is conjugated or linked to the scaffold nucleic acid molecule. In certain embodiments, the vaccine adjuvant is conjugated or linked to a complementary nucleic acid molecule. In certain embodiments, the scaffold nucleic acid molecule and/or complementary nucleic acid molecule further comprises a DNA-based adjuvants (e.g., CpG adjuvants). In certain embodiments, the vaccine adjuvant is contained within a composition comprising the immunogenic construct and a carrier (e.g., a pharmaceutically acceptable carrier).

In certain embodiments, the immunogenic construct of the instant invention further comprises a substrate which improves cellular uptake and/or biological stability. Examples of such substrates include, without limitation: nanoparticles, microparticles, dendrimers, nucleic acid origamis, virus-like particles, recombinant proteins, polysaccharides, and combinations thereof. Examples of nanoparticles include, without limitation: metallic-based nanoparticles, carbon-based nanoparticles, polymeric nanoparticles, lipid-based nanoparticles, and ceramic nanoparticles. In certain embodiments, the substrate is a metallic-based nanoparticle, particularly a gold nanoparticle.

In certain embodiments, the scaffold nucleic acid molecule (e.g., at the 3′ or 5′ end) is conjugated or linked to a nanoparticle, particularly a metal or gold nanoparticle. The scaffold nucleic acid molecule may comprise a thiol group (—SH) for conjugation to the nanoparticle (e.g., metal or gold nanoparticle). For example, the scaffold nucleic acid molecule may further comprise a linker with a terminal thiol group. The linker may be a nucleic acid molecule (e.g., DNA (e.g., an adenosine stretch)). For example, the linker may be 1-25 nucleotides, 1-20 nucleotides, 1-15 nucleotides, 5-20 nucleotides, or 5-15 nucleotides in length. The thiol functionalized scaffold nucleic acid molecule may then be conjugated to the nanoparticle (e.g., metal or gold nanoparticle). In certain embodiments, the thiol functionalized scaffold nucleic acid molecule is conjugated to the nanoparticle (e.g., metal or gold nanoparticle) by a freezing-based conjugation (e.g., freezing and thawing the immunogenic construct with the nanoparticle). In certain embodiments, the thiol functionalized scaffold nucleic acid molecule is conjugated to the nanoparticle (e.g., metal or gold nanoparticle) by a pH-assisted conjugation (e.g., reducing the pH to about 3 and neutralizing prior to heat annealing).

While the scaffold nucleic acid molecules and complementary nucleic acid molecules of the instant invention are generally described as DNA herein, the nucleic acid molecules may comprise DNA-like complementary polymers and/or comprise nucleic acid analogs. Similarly, nucleic acid linkers may comprise DNA-like complementary polymers and/or comprise nucleic acid analogs. For example, the scaffold nucleic acid molecules and/or complementary nucleic acid molecules of the instant invention may comprise one or more of DNA, RNA, 2′-F nucleic acids, 2′-oxyalkyl nucleic acids, 2′ bridged (LNA) nucleic acids, mirror nucleic acids (L-DNA and L-RNA), threose nucleic acids, unlocked (acyclic) nucleic acids, phosphorothioate nucleic acids, peptide nucleic acids, morpholino oligonucleotides, and the like. The nucleic acids may also comprise nucleobase analog pairs including, but not limited to: diaminopurine-thymine and isoguanine-isocytosine. In certain embodiments, the nucleic acid molecules may be referred to as oligonucleotides.

The programmable complementarity of nucleic acids and analogs permits orthogonal assembly of multiple moieties in bioconjugation chemistry-independent manner, with precisely defined spatial arrangement of the ligands on the scaffold. The nucleic acid analogs may be used to modulate the biological activity and circulation half-life of the self-assembled immunogen. For example, mirror nucleic acids are biologically inert and have long circulation half-life, while phosphorothioate nucleic acids are potent immune stimulators with long circulation half-life.

The self-assembly of nucleic acids and analogs is a highly specific, robust process with high fidelity, and enables assembly of a wide variety of shapes and structures. This unique aspect of nucleic acids and analogs enables assembly of ligands into higher order ligand assemblies, such as conformational and discontinuous epitopes. This enables the invention to be used for immunotherapy applications in diseases mediated by the assembly proteins into aggregates, such as Alzheimer's disease, as well as for the generation of vaccines against cryptic neutralizing epitopes on pathogens of infectious diseases such as malaria, HIV, and various respiratory diseases.

The present invention also encompasses compositions comprising at least one immunogenic construct of the instant invention and at least one carrier (e.g., a pharmaceutically acceptable carrier). The compositions of the instant invention can be used in medicine.

The present invention also encompasses methods for preventing, inhibiting, and/or treating a disease or disorder in a subject. The compositions of the instant invention can be administered to an animal, in particular a mammal (e.g., a human) in order to treat, inhibit, and/or prevent a disease or disorder. In certain embodiments, the disease or disorder is characterized by a pathogenic protein. In certain embodiments, the disease or disorder is cancer. In certain embodiments, the disease or disorder is Alzheimer's disease. In certain embodiments, the disease or disorder is caused by a pathogen. In certain embodiments, the disease or disorder is caused by a viral infection, such as SARS-COV-2.

As stated hereinabove, the present invention also encompasses compositions comprising at least one immunogenic construct of the instant invention and at least one carrier (e.g., pharmaceutically acceptable carrier). The compositions comprising the immunogenic construct of the instant invention may be conveniently formulated for administration with any pharmaceutically acceptable carrier(s). For example, the immunogenic construct may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the immunogenic construct in the chosen medium may be varied and the medium may be chosen based on the desired route of administration of the pharmaceutical composition. Except insofar as any conventional media or agent is incompatible with the immunogenic construct to be administered, its use in the pharmaceutical composition is contemplated.

The immunogenic constructs described herein will generally be administered to a patient as a pharmaceutical composition. The term “patient” as used herein refers to human or animal subjects. These immunogenic constructs may be employed therapeutically, under the guidance of a physician.

The dose and dosage regimen of immunogenic constructs according to the invention that are suitable for administration to a particular patient may be determined by a physician considering the patient's age, sex, weight, general medical condition, and the specific condition for which the immunogenic constructs are being administered and the severity thereof. The physician may also take into account the route of administration, the pharmaceutical carrier, and the immunogenic construct's biological activity.

Selection of a suitable pharmaceutical composition will also depend upon the mode of administration chosen. For example, the immunogenic construct of the invention may be administered by direct injection, intraperitoneally, or intravenously. In this instance, a pharmaceutical composition comprises the immunogenic construct dispersed in a medium that is compatible with the site of injection.

Immunogenic constructs of the instant invention may be administered by any method. For example, the immunogenic constructs of the instant invention can be administered, without limitation by injection, parenterally, subcutaneously, orally, nasally, topically, pulmonarily, rectally, vaginally, intravenously, intraperitoneally, intrathecally, intracerbrally, epidurally, intramuscularly, intradermally, intracarotidly, or other modes of administration such as controlled release devices. In a particular embodiment, the immunogenic constructs (or compositions) are administered intramuscularly, subcutaneously, or to the bloodstream (e.g., intravenously). In a particular embodiment, the immunogenic constructs (or compositions) are administered by injection. In general, pharmaceutical compositions and carriers of the present invention comprise, among other things, pharmaceutically acceptable diluents, preservatives, stabilizing agents, solubilizers, emulsifiers, adjuvants and/or carriers. Such compositions can include diluents of various buffer content (e.g., saline, Tris HCl, acetate, phosphate), pH and ionic strength; and additives such as detergents and solubilizing agents (e.g., Tween™ 80, polysorbate 80), anti-oxidants (e.g., ascorbic acid, sodium metabisulfite), preservatives (e.g., Thimersol, benzyl alcohol) and bulking substances (e.g., lactose, mannitol). The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. Exemplary pharmaceutical compositions and carriers are provided, e.g., in “Remington's Pharmaceutical Sciences” by E. W. Martin (Mack Pub. Co., Easton, Pa.) and “Remington: The Science and Practice Of Pharmacy” by Alfonso R. Gennaro (Lippincott Williams & Wilkins) which are herein incorporated by reference.

Pharmaceutical compositions for injection are known in the art. If injection is selected as a method for administering the immunogenic construct, steps should be taken to ensure that sufficient amounts of the molecules or constructs reach their target cells to exert a biological effect.

Pharmaceutical compositions containing an immunogenic construct of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, topical, oral, direct injection, intracranial, and intravitreal.

The instant invention also encompasses methods of producing the immunogenic construct. In certain embodiments, the method comprises hybridizing the complementary nucleic acid molecules to the scaffold nucleic acid molecule. In certain embodiments, the method further comprises conjugating the peptide to the complementary nucleic acid molecule prior to hybridization. In certain embodiments, the method further comprises synthesizing the nucleic acid molecules and/or peptides. In certain embodiments, the method further comprises conjugating the immunogenic complex to a nanoparticle, particularly a gold nanoparticle. In certain embodiments, the conjugation to the nanoparticle is performed by pH assisted conjugation. In certain embodiments, the conjugation to the nanoparticle is performed by freezing based conjugation.

### Definitions

The following definitions are provided to facilitate an understanding of the present invention.

The singular forms “a,” “an,” and “the” include plural referents unless the context clearly dictates otherwise.

The term “isolated” is not meant to exclude artificial or synthetic mixtures with other compounds or materials, or the presence of impurities that do not interfere with the fundamental activity, and that may be present, for example, due to incomplete purification, or the addition of stabilizers.

The term “substantially pure” refers to a preparation comprising at least 50-60% by weight of a given material (e.g., nucleic acid, oligonucleotide, protein, etc.). Particularly, the preparation comprises at least 75% by weight, at least 80% by weight, at least 90% by weight, or at least 95% or more by weight of the given compound. Purity may be measured by methods appropriate for the given compound (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween™ 80, polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., an infection, disease or disorder) resulting in a decrease in the probability that the subject will develop the condition.

A “therapeutically effective amount” of a compound or a pharmaceutical composition refers to an amount effective to prevent, inhibit, treat, and/or lessen the symptoms of a particular disorder or disease. The treatment of a viral infection herein may refer to an amount sufficient to inhibit viral reproduction and/or curing, relieving, and/or preventing the viral infection, the symptom of it, or the predisposition towards it.

As used herein, the term “subject” refers to an animal, particularly a mammal, particularly a human.

The following examples describe illustrative methods of practicing the instant invention and are not intended to limit the scope of the invention in any way.

### Example 1

Vaccination is considered the most efficient countermeasure for the current COVID-19 pandemic caused by SARS-COV-2. The current strategies for vaccine development include approaches utilizing inactivated virus, live attenuated virus, virus-like particles, full or subdomain sized viral proteins, recombinant DNA vectors, and mRNA in lipid nanoparticles. Each of these approaches has advantages and disadvantages. Herein, a novel vaccine platform approach to treat the COVID-19 pandemic is provided. The vaccine is based on the rational design of an immunogen containing several defined B-cell epitopes to elicit highly specific neutralizing immunity, while minimizing off-target reactions and preventing vaccine-induced enhancement of the infection. Each epitope is a relatively short peptide synthesized chemically and identified based on the validated bioinformatic data about the target. The assembly was tested with the use of two peptides from the spike(S) protein of SARS-COV2 serving as B-cell epitopes for the virus (Li et al., Cellular & Molecular Immunology (2020) 17:1095-1097). The peptides are selected from neutralizing linear B-cell epitopes. These peptides can be synthesized chemically, so any needed post-translational modification can be included in the peptide sequence.

### Materials and Methods

### Vaccine Preparation

Following N-terminal azidolysine-labeled peptides at 95% purity were purchased from Genscript (Piscataway, NJ).
1. P1 is the pan HLA DR-binding epitope (PADRE) and is designed to stimulate T helper cells in genetically diverse populations. PADRE is a universal T helper epitope capable of inducing strong T cell response in the majority of T cell genotypes (Alexander, et al., J. Immunol. (2000) 164 (3): 1625-1633). The peptide sequence is (LysN3)-GSAKFVAAWTLKAAA (SEQ ID NO: 1).
2. P2 is derived from residues 1148-1159 of the full spike. This region is located right before the heptad repeat 2, which is important for membrane fusion (e.g., HR2 interacts with HR 1 and induces fusion). The peptide sequence is (LysN₃)-FKEELDKYFKNH (SEQ ID NO: 2).
3. P3 is derived from residues 553-564 of the full spike. This region is located on the S1, right after the receptor binding domain (RBD) involved in conformational transition of S-protein necessary for receptor recognition. The peptide sequence is (LysN₃)-TESNKKFLPFQQ (SEQ ID NO: 3).

The DNA oligonucleotides (ssDNA) were synthesized using a MerMade12 DNA synthesizer with the help of the standard phosphoramidite chemistry. The following set of oligonucleotides was synthesized:

Strand 1
(SEQ ID NO: 4)
DBCO-TATACAGCCTACTCACTATA for coupling with P1;
Strand 2
(SEQ ID NO: 5)
DBCO-TATACTGAGCTAGTCGTATA for coupling with P2;
Strand 3
(SEQ ID NO: 6)
DBCO-TATACCTTCATCCTTATATA for coupling with P3;
Scaffold strand:
(SEQ ID NO: 7)
(SH)-A₉-TATAGTGAGTAGGCTGTATA-
TATACGACTAGCTCAGTATA-TATATAAGGATGAAGGTATA.

Citrate-coated gold nanoparticles, 15 nm in diameter and at optical density (OD) 50 were purchased from Luna Nanotech (Markham, ON, Canada) and used for formulation.

All materials were purchased pyrogen-free and sterile and handled aseptically.

### Peptide-DNA Coupling

Each peptide and DNA strand were dissolved in 6 M guanidinium hydrochloride in the presence of sodium phosphate buffer (50 mM, pH 7.0) at the final concentrations of 200 μM ssDNA and 400 μM peptides. The reaction was carried out for 24 hours at 4° C., followed by dialysis to remove excess peptides. Reaction completion was verified by the disappearance of 308 nm absorbance shoulder on UV-Vis, corresponding to the unreacted DBCO. Coupling and dialysis were performed aseptically.

### Assembly of the Antigen

Peptide-coupled DNA strands (1-3) were combined in 0.5×PBS pH 7.4 at a concentration of 150 UM and 140 μM of the complement. The mixture was heated to 95° C. and gradually cooled to 4° C. (2 hours). The annealed product was diluted to 1.2 OD at 260 nm in isotonic PBS. These steps were performed aseptically. The assembly was verified by the melting experiments using UV-Vis spectrophotometer Varian Cary 50 Bio (Varian Palo Alto, CA) with a temperature range of 10-85° C., data interval 0.1° C., temperature ramp rate 2° C./minute, signal averaging time 0.1. The melting temperature was compared to the theoretically melting temperature predicted by OligoAnalyzer™ from Integrated DNA Technologies (IDTDNA.com).

### Vaccine Formulation

Vaccine candidates were formulated by the following four methods:

In the freezing-based conjugation (FR) method, the annealing product was diluted in water 4-fold (<20 mM Na+) and added to the gold nanoparticles (GNP) stock solution (136 nM) at a molar ratio of 150:1. The mixture was placed in a −20° C. freezer for 1 hour. Then, the product was allowed to thaw, and 10×PBS was added to adjust the tonicity of the final product to isotonic. The solution was diluted to 50 nM (as GNP) in PBS, aliquoted at 100 μl/dose, and frozen for use as an immunogen.

In the pH-assisted conjugation (PA) method, the annealing product was combined with GNP stock solution at a molar ratio of 50:1 and incubated for 5 minutes. Then, pH was adjusted to 3.0 with 1 M citric acid, incubated for 3 minutes, and neutralized with NaOH to pH 6.5. Heat annealing was performed as described. The conjugate was dialyzed against isotonic PBS, diluted, and aliquoted as in the FR method.

In the salt concentration-based conjugation (SC) method, the initial procedure was identical to the FR method. Instead of freezing, saturated NaCl (6.15 M) was gradually added to a 1 M final concentration (48 hours). The conjugate was dialyzed against isotonic PBS using a 50 kDa molecular weight cut-off membrane, diluted, and aliquoted as in the FR method.

For gold-free inulin (IN) co-crystallization method, inulin microcrystals (epsilon form) were prepared as described (Cooper, et al., Glycobiology (2013) 23 (10): 1164-1174). The microcrystals were washed five times via centrifugation and resuspension in 45° C. water. After the final centrifugation, the wet pellet was resuspended in the annealed product at final proportions of 300 mg of wet pellet and 5 nmol of annealed product per 1 ml isotonic PBS. The mixture was then heated up to 55° C. and allowed to cool down slowly. The resulting microcrystals were aliquoted at 100 μl/dose and stored at 4° C. until further use.

All formulation-related procedures were performed aseptically.

### Agarose Gel Electrophoresis

Agarose (2%) (Sigma-Aldrich, Inc. St. Louis, MO) gel was prepared in sodium borate buffer (10 mM, pH 8.0) and used for electrophoresis of unconjugated and conjugated GNP prepared by the FR, PA, and SC methods. The nanoparticles were loaded directly in buffer without loading dye at 10 μl/well, and electrophoresis was carried out at 15 V/cm (300 V).

### AFM Imaging

Samples for AFM scanning were diluted for each conjugation method to 0.5 nM before depositing onto 1-(3-aminopropyl)-silatrane (APS) functionalized mica. The samples were incubated for 2 minutes to bind to the APS mica, followed by gentle washing with DI water and drying with a slow argon gas flow. The samples were then dried in a vacuum chamber overnight.

Once dried, the samples were scanned on a Multimode AFM/Nanoscope IIId system with TESPA probes (Bruker Nano Inc., Camarillo, CA). The images captured were 3×3 μm in size with 1536 pixels/line.

The images were analyzed using Femtoscan software (Advance Technologies Center, Moscow, Russia). A cross-sectional line was drawn over the center of each particle and a diameter in nm was obtained. Once all the particles on each image were analyzed, histograms were generated using Origin software.

### Dynamic Light Scattering

The Dynamic Light Scattering (DLS) and zeta potential scans were completed on a Malvern NanoZS meter. The DLS samples were diluted 100× (0.5 nM) before filling the cuvette for DLS measurements. The computer software from Malvern Instruments, Ltd., automatically generated the graphs. The DLS experiments were completed in triplicate, and the runs were overlayed on the same graph. The Zeta potential measurement samples were diluted 50×(1 nM), and the Malvern Instruments, Ltd., software fitted the different zeta potential on an aggregate graph, allowing for easy comparison between the ten different runs. If a run was an extreme outlier from the other runs, it was excluded from the results.

### Cytotoxicity Assay

Vero-E6 cells were seeded at 10,000 cells/well in a 96-well plate containing 100 μl complete media specific for each cell type. For adherence, cells were incubated overnight at 37° C. in a humidified 5% CO₂ incubator. After overnight incubation, the media was replaced with fresh media, and Vero-E6 cells were treated with the formulations at 50 nM with or without adjuvant. Untreated cells were considered a negative control. After the treatment, cells were incubated at 37° C. in humidified 5% CO₂ incubator. 48 hours post-treatment, 20 μl of MTT substrate (5 mg/ml) was added to each well and incubated for 4 additional hours at 37° C. in the dark. Then the culture media was carefully removed, the blue formazan crystals were dissolved in 200 μl of DMSO, and the purple color was read at 590 nm with a reference filter of 620 nm.

### Cells and Viral Titer Determination

Vero E6 (ATCC® CRL-1586™) and Vero-STAT1 knockout cells (ATCC® CCL-81-VHG™) were cultured in DMEM containing 10% fetal bovine serum, 2 mM 1-glutamine, penicillin (100 units/ml), streptomycin (100 units/ml), and 10 mM HEPES. Calu-3 cell (ATCC 184HTB-55) were cultured in Eagle's 188 Minimum Essential Medium (EMEM) (ATCC 30-2003) containing 10% FBS. SARS-COV-2 isolates USA-WI1/2020 (BEI; cat #NR-52384), USA-WA1/2020 (BEI; cat #NR-NR-52281) were passaged in Vero-STAT1 knockout cells, whereas hCoV-19/USA/PHC658/2021 (Delta Variant) (BEI; cat #NR-55672) was passaged in Calu-3 cells. The viral titer was determined using the plaque assay as described (Mendoza, et al., Curr. Protoc. Microbiol. (2020) 57 (1): cpmc105). In brief, Vero E6 cells (2.5×10⁵) were seeded in 6-well plates and incubated for 24 hours. After 24 hours, cells were washed with sterile 1×PBS, and the virus stock was ten-fold serially diluted in serum-free OptiMEM™ media and then added to the cells in duplicate. The plates were incubated at 37° C. for 1 hour with slight shaking every 15 minutes. Then, 2 ml of 0.5% agarose in minimal essential media (MEM) containing 5% FBS and antibiotics, penicillin (100 units/ml), and streptomycin (100 units/ml), were added to each well and incubated at 37° C. for 72 hours. The cells were fixed with 4% paraformaldehyde overnight, followed by removing the overlay and staining with 0.2% crystal violet to visualize plaque-forming units (PFU). All assays were performed in a BSL-3 laboratory setting.

### Animal Experiments

BALB/c mice (males, 6 to 8 weeks old) were purchased from Jackson Laboratories, housed in micro isolator cages, and maintained at 12-hour light-dark cycle at 22.2° C., and 30-40% humidity. Mice were given feed and sterile water daily, and they were acclimated to the environment for approximately 1-2 weeks to determine that they were healthy and suitable for experiments. Four groups of mice (n=5 each group) were immunized subcutaneously with GNP-conjugated vaccines (100 μl) by different methods, FR, PA, SC, and IN. The immunogens were diluted in LPS-free water and mixed with 25 μg ODN 1826 Class B CpG oligonucleotide (a murine TLR9 ligand) as an adjuvant. Another group of mice immunized with only 25 μg ODN 1826 Class B CpG oligonucleotide serves as the control. Baseline blood samples were collected from the submaxillary vein on day 0 (before the immunization). Blood samples were collected every 14 days with boosts of the same doses were given. All mice were immunized with 2 boosts and finally necropsied at day 61. Blood samples and spleens were collected at necropsy for flow cytometry.

### Enzyme-Linked Immunosorbent Assay (ELISA)

Ninety-six-well high binding plates were separately coated with peptide P1, P2 (100 ng/well), whole spike, and spike-RBD glycoproteins (0.1 μg/well) 1× phosphate buffered saline (PBS) and incubated at 4° C. overnight. The next day, the plates were washed three times and blocked with 1% BSA in 1×PBS containing 0.1% Tween® 20 (PBST) for 1 hour at 37° C. Sera samples were diluted five-fold serially starting at 1:25 in 1% BSA containing PBST, then added to the plates and incubated at 37° C. for 1 hour. The plates were washed three times with PBST, then HRP-conjugated goat anti-mice IgG Human ads-HRP (SouthernBiotech1:4,000 dilution) (cat #103005) was added and incubated for 1 hour at room temperature. Plates were washed five times with PBST before adding 3,3′,5,5′-tetramethylbenzidine (TMB) substrate solution. The reaction was stopped after 5 minutes by adding 0.16 M sulfuric acid. The OD at 450 nm was measured with a Bio-Rad microplate reader. A graph was plotted as Absorbance at 450 nm vs. dilutions.

### Immunofluorescence-Based Neutralization Assay

Neutralization assays were performed against USA_WI/2020, USA-WA1/2020, and hCoV-19/USA/PHC658/2021 strains. Sera samples were serially diluted starting from 1:25 (5-fold) in serum-free Dulbecco's modified Eagle's medium (DMEM) in triplicate wells and incubated with 20,000 focus-forming units of SARS-COV-2 virus at 37° C. for 1 hour. The serum-virus mixture was added to Vero E6 cell (C1008, ATCC, no. CRL-1586) monolayers seeded in 96 healthy blackout plates and incubated at 37° C. for 1 hour. The inoculum was removed and replaced with complete DMEM and incubated at 37° C. for 24 hours. After 24 hours, DMEM was removed, and cells were washed twice with PBS and fixed with 4% paraformaldehyde in PBS for 30 minutes at room temperature. Following fixation, plates were washed thrice with 1×PBS and permeabilized with 50 μl/well of 0.1% Triton™ X-100 (Fisher BP151-100) in PBS for 10 minutes. After permeabilization, cells were washed thrice with 1×PBS and blocked with 3% BSA for 30 minutes. Then, cells were stained with 50 μl/well of primary antibody, anti-SARS-COV-2 spike (rabbit mAb, Sino Biologicals MA14AP0204) at 1:1000 diluted in 3% BSA-PBS and incubated overnight at 4° C. on a shaker. The next day, cells were washed three times with 1×PBS and stained with a secondary antibody (Alexa Fluor® 488 Goat anti-rabbit) at 1:2000 dilution in 3% BSA-PBS. Finally, cells were incubated at room temperature in a shaker for 1 hour, washed three times with 1×PBS, and stained the nuclei using Hoechst 33342 (Invitrogen H3570) and Cell Mask (Invitrogen C10046) at 1:20,000 diluted in 1×PBS. The plate was shaken for 15 minutes after proper sealing with aluminum foil/sealer and taken for reading using Operetta Imager. Percentage neutralization was calculated based on the difference in fluorescent intensity.

### Analysis of B Cells and CD4+/CD8+ Memory Phenotypes by Flow Cytometry

Blood samples and spleen tissues were collected from immunized mice at necropsy. Single-cell suspension from spleen tissues was prepared, and RBCs were lysed using RBC lysis buffer. Cells (5×10⁶) were washed and suspended in staining buffer, and Fc block was performed with purified anti-mouse CD16/32. Dead cells were discriminated by adding Zombie Aqua™ fixable viability dye. Fluorochrome-labeled mouse-specific antibodies were diluted with staining buffer into cocktails and added to cells at 100 μl per sample. Cells were incubated for 30 minutes at room temperature and washed twice with staining buffer. Then, cells were fixed using 2% PFA and acquired using the Fortessa™ X450 flow cytometer (Becton Dickson). Fluorescence minus one (FMO) control was performed in parallel, and FMO determined subsequent gating. All data were analyzed using Flowjo version 10.6 (Trees Star Inc., Ashland, OR) software.

### Statistical Analysis

Two-way ANOVA with Tukey's multiple comparison test was performed to assess statistical significance (P values <0.05 is considered significant) using (GraphPad PRISM 6.07, San Diego, CA) software.

### Results

### Preparation of the Vaccine

The immunogen is prepared from three synthetic peptides, three short DNA strands (probes), and one long DNA strand, which is complementary to the three probes and acts as a sequence-specific scaffold for assembly.

Each DNA strand was synthesized chemically. A copper-free click chemistry dibenzocyclooctyne (DBCO) handle is introduced at the 5′ of each probe strand for attachment to the epitopes, and a thiol is introduced to the 5′ of the scaffold for downstream use during formulation.

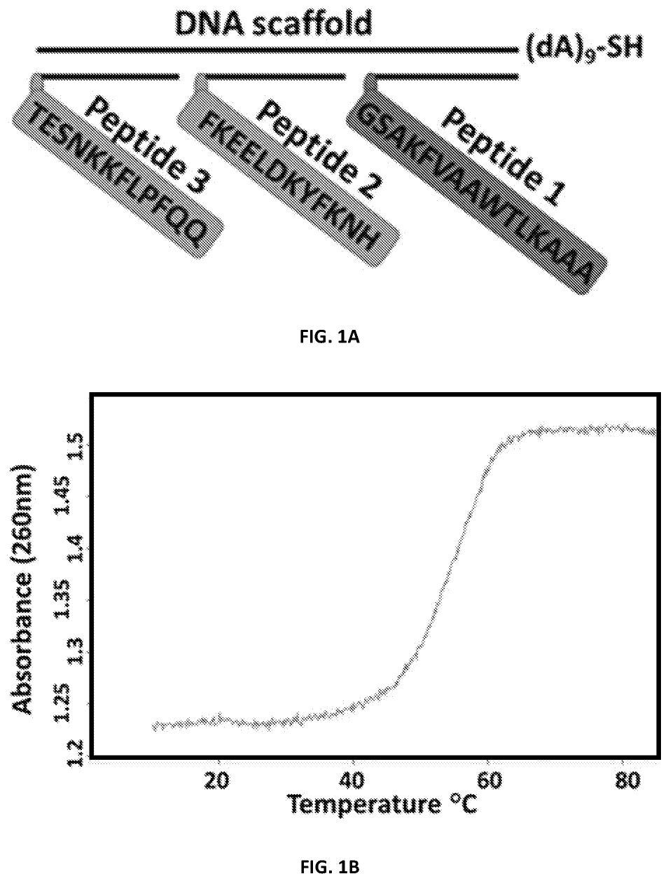

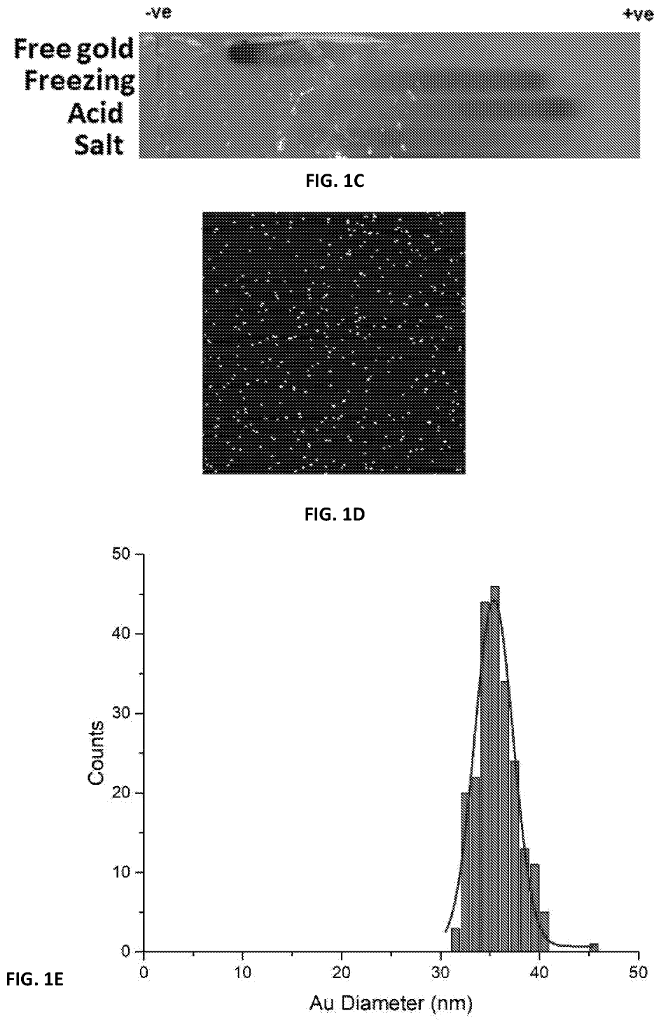

B-cell epitopes (peptides P2 and P3) are selected from the reported linear neutralizing epitopes of the SARS-COV-2 spike (Li, et al., Cell Mol. Immunol. (2020) 17 (10): 1095-1097). The T-cell epitope is the artificial universal T helper epitope PADRE (peptide P1), ensuring robust immunostimulatory signaling (Rosa, D., Immunol. Lett. (2004) 92 (3): 259-268). Here, the peptides are synthesized with an N-terminal azidolysine for conjugation to the appropriate DBCO-labeled probes via click chemistry. The resulting probe-epitope conjugates and the scaffold DNA are the individual modules of our immunogen. Upon equimolar mixing, they spontaneously assemble into the immunogen by sequence-specific hybridization of the probes with the corresponding regions of the scaffold. The resulting immunogen complex is shown in FIG. 1A.

The assembly was validated by UV spectrophotometric melting experiments. The melting profile for the construct is shown in (FIG. 1B). The sharp increase in absorbance near 55° C. corresponds to the experimental melting temperature. This confirms the presence of a DNA duplex, and the absence of a hyperchromic effect at temperatures below 40° C. confirms stability at physiological temperatures.

### Selecting the Optimal Method for Formulation

Conjugation to gold nanoparticles (GNP) was utilized to improve the uptake and biological stability of the construct (Mateu Ferrando, et al., Drug Discov. Today Technol. (2021) 38:57-67). Three methods were used to conjugate the immunogen to GNP: a) freezing-based conjugation (FR), b) pH-assisted conjugation (PA), and c) salt concentration-based conjugation (SC). The immunogen assembly binds to the nanogold via the formation of strong bonds between the top DNA strand of the immunogen and gold atoms of the nanoparticle forming a corona around the nanoparticle.

The GNP-immunogen formulations are negatively-charged particles with the net charge of particles defined by the density of coverage of the nanoparticle with immunogens. Therefore, gel electrophoresis was used to characterize the formulations (FIG. 1C). The PA formulation exhibited the highest mobility towards the positive electrode, followed by the FR formulation, which had slightly lower mobility. The SC formulation produced a diffuse band with low electrophoretic mobility. These results indicated that the immunogen conjugation with FR and PA methods produced particles with a higher immunogen density per GNP than the SC method. A fourth gold-free formulation (IN) based on co-crystallization with microcrystalline inulin was chosen since inulin is known to activate complement and improve antigen uptake (Cooper, et al., Glycobiology (2013) 23 (10): 1164-1174). This formulation served as a control for the GNP formulations.

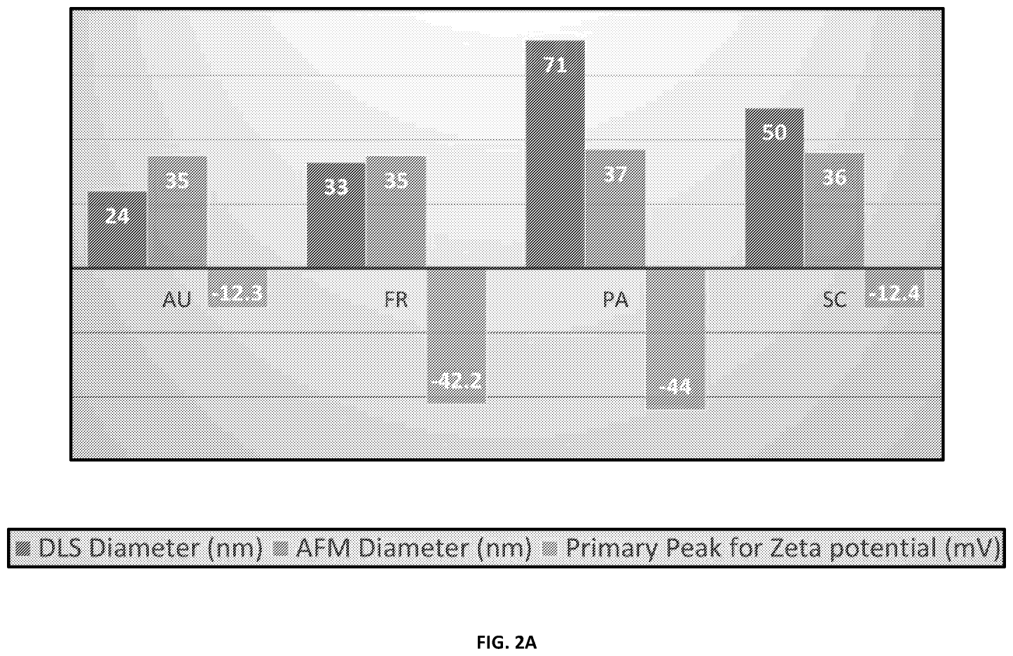

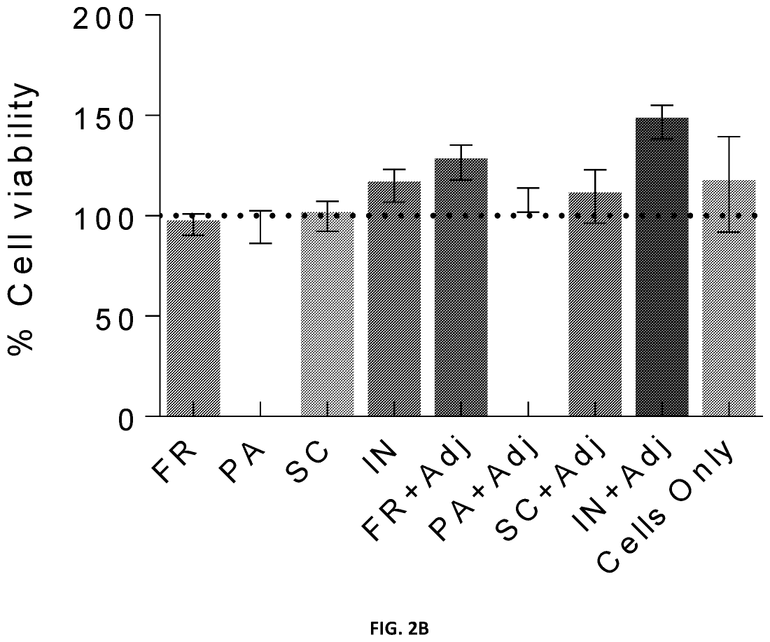

AFM topographic imaging and dynamic light scattering (DLS) was used to provide additional characterization of the vaccine formulations. In the AFM experiments, the samples of formulations, along with the unconjugated gold nanoparticles used as a control, were diluted to the concentration 0.5 nM, deposited on functionalized mica (Shlyakhtenko, et al., Cell Imaging Techniques (2012) Humana Press, pages 295-312) (APS-mica) for 2 minutes, rinsed with deionized water, dried, and imaged with AFM operating in tapping mode. Images of gold nanoparticles are shown in FIG. 1D. Spherical particles are clearly seen in the images. The sizes of each particle (diameter) were measured using the cross-section tool of the AFM software. The histogram of the diameter distribution is shown in FIG. 1E. The histogram is fit by a Gaussian with the maximum 35.4 nm±0.1 nm (SEM). The results of DLS data for the same sample produced a mean value of 24 nm. Similar characterization was done for the vaccine formulations. The measurements for all samples are combined as histograms in FIG. 2A.

The results demonstrate that the conjugation method using PA resulted in the largest DLS diameter of 71 nm, followed by SC at 50 nm, FR at 33 nm and Au (gold nano particles) at 24 nm. The PA conjugation method also resulted in the greatest negative value zeta potential. AFM data compared with the DLS ones show lower values for particle sizes, which is consistent with the drying of the sample for AFM measurements. The Zeta potentials for PA and FR formulations illustrate higher values than the control, which is consistent with the gel-electrophoresis data illustrating the negative charge increase by coating with the negatively charged DNA backbone of the vaccine.

The cytotoxicity of the samples was also tested. The results are assembled in FIG. 2B. The cytotoxicity of the four formulations was measured in Vero-E6 cells with or without adjuvant using a standard colorimetric MTT assay that measures cell proliferation and viability twice. The percent viability of the cells is plotted. All the conjugated immunogens have minimal or <10% cytotoxicity in Vero-E6. The CpG adjuvant-only treated cells have zero toxicity in Vero-E6 cells.

These four immunogen formulations were further tested for their ability to induce an antigen-specific immune response in mice.

### Comparing the Formulations Based on the Peptide-Specific IgG Response Strengths

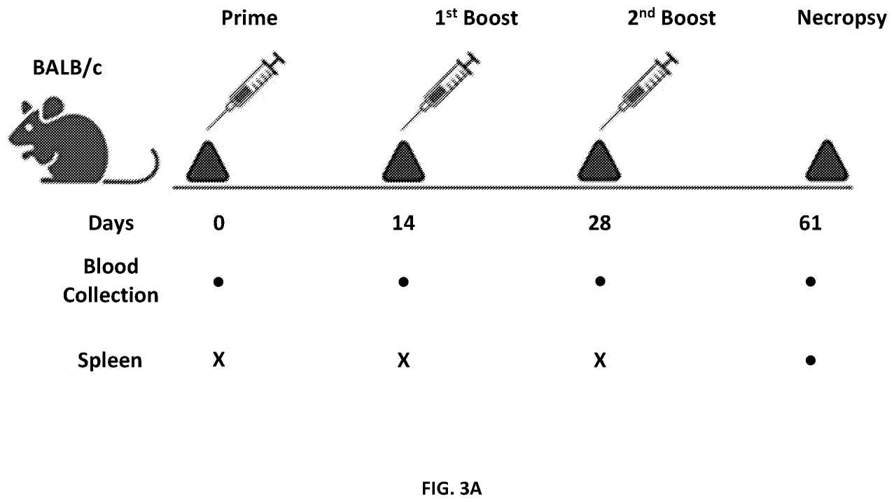

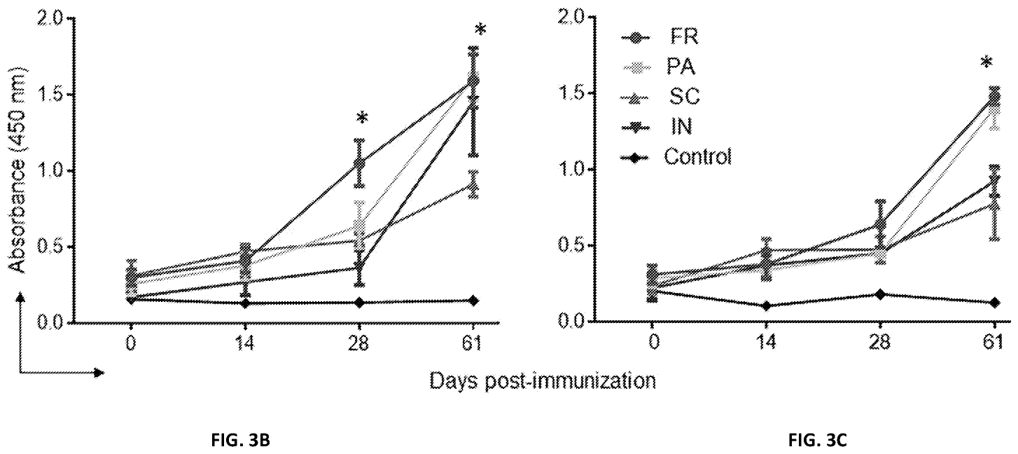

Four groups of BALB/C mice were immunized with the FR, PA, SC, and IN immunogen formulations (25 mg/injection) with a class B CpG adjuvant (100 μl/injection). A control group of mice received the adjuvant alone. All five groups of mice were immunized with three doses of the respective immunogen as prime dose (0 days), 1st boost (14 days,) and 2nd boost (28 days), as shown in schema (FIG. 3A). Sera were collected on days 0, 14, 28, 61, and tested by ELISA for IgG against peptides P1 and P2, as well as the whole protein S or its receptor-binding domain (RBD) fragment. Plates were coated separately with P1 or P2 (0.1 μg/well) to determine the peptide-specific IgG response. Sera from each time point were applied in 5-fold serial dilutions from 1:25 to 1:78125. Sera from all the four vaccinated groups (FR, PA, SC, and IN) indicated the presence of detectable IgG immune response against P1 and P2 at 1:25 on day 61 (FIG. 3B, 3C). However, mice immunized with immunogens formulated by FR and PA methods elicited higher IgG immune response with a maximal immune response on day 61. The immunogen formulated by the SC method elicited a lower IgG immune response against both P1 and P2 (FIG. 3B, 3C). On the other hand, mice immunized with immunogen formulated with IN exhibited a higher IgG immune response against P1 than against P2 (FIG. 3B, 3C). The observed difference in IgG immune response to immunogens produced by FR and PA methods versus SC method correlated with the density of immunogen on the gold nanoparticles produced by each method (FIG. 3C). These results indicate that GNP-conjugated immunogens with high-density coverage of the nanoparticles elicit a stronger response, while the inulin formulation does not efficiently co-deliver the P1 and P2, leading to poor T helper activation of P2 specific B cells.

### Binding of the Whole Spike in ELISA

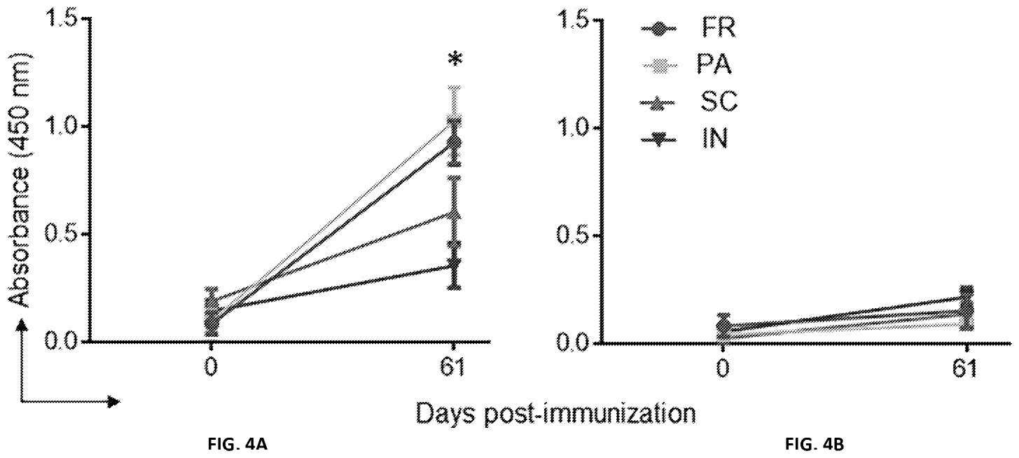

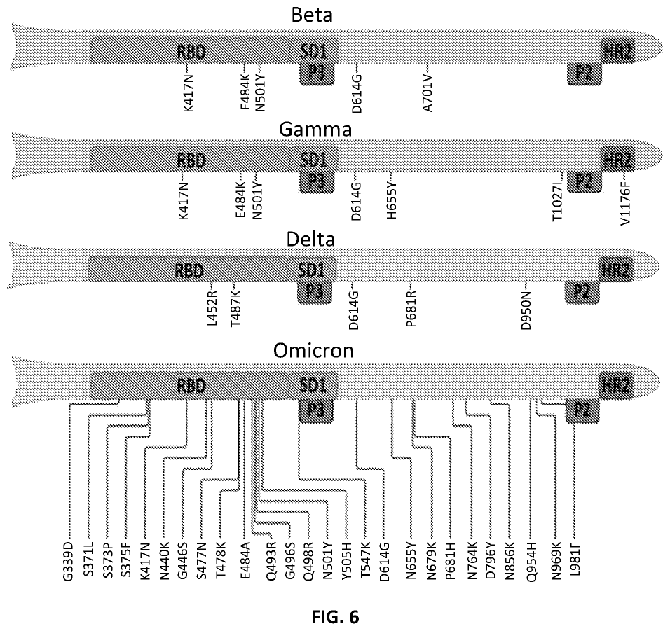

To confirm that the peptide-specific antibodies can bind the same regions in the native spike, ELISA was used with plates coated with stabilized whole spike glycoprotein. The receptor-binding domain (RBD) protein was used as a negative control since it includes neither of our peptides in its sequence. The sera were applied in 5-fold serial dilutions, starting with 1:25 and up to 1:78125. The immune responses were similar to the previous results against P2. FR and PA immunogens elicited a higher immune response against spike after the 2nd booster at 1:25 dilutions (FIG. 4A). SC formulation elicited a detectable but far weaker IgG immune response. In contrast, the IN formulation had comparable signals against negative control (RBD) and whole spike (FIG. 4B). The position of the P2 and P3 on the spike are depicted in FIG. 6. Subdomain 1 (SD1) follows the receptor-binding domain (RBD) and is essential for conformational transitions responsible for recognition by cellular receptors. Heptad repeats 1 and 2 (HR1, HR2) interact to form a six-helix bundle responsible for the fusion of the viral and cellular membranes. Selected epitopes disrupt both processes, thus preventing attachment to the cell membrane and subsequent fusion.

Virus Neutralization with the Generated Sera

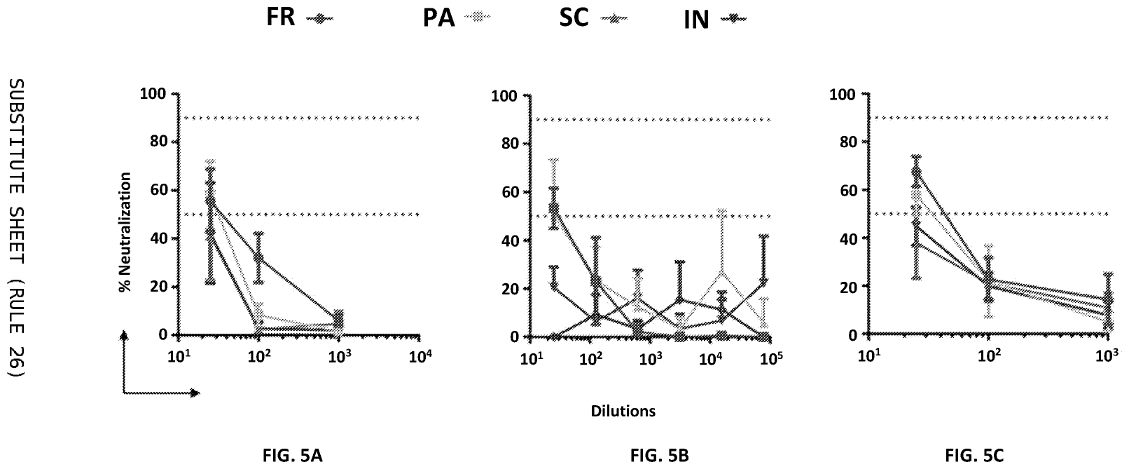

The neutralization activity of pooled sera from each group was tested against three SARS-COV-2 strains: SARS-COV-2 isolate USA-WA1/2020 (FIG. 5A), SARS-CoV-2 isolate USA-WI1/2020 (FIG. 5B), and SARS-COV-2 isolate hCoV-19/USA/PHC658/2021 (Delta Variant) (FIG. 5C). It was found that at 1:25 dilution, total sera from PA and FR groups could neutralize both wild-type and delta variants of SARS-COV-2 (>50% neutralization), and higher dilutions of sera showed <50% neutralization titers (FIG. 5). These data indicate that GNP-conjugated vaccine candidates elicit antigen-specific immune responses with neutralization potential.

### Flow Cytometry Analysis of CD20+ B Cells and CD4+CD8+ T Cells Memory Phenotypes in GNP-Conjugated Vaccine Groups

Using a flow cytometric analysis, a phenotypic assessment of CD4+ and CD8+ T cells and B cells in the spleen and peripheral blood in control and GNP-conjugated vaccine immunized mice at necropsy (Day 61) were performed. Here, CD4+ and CD8+ T cells phenotypic assessment was performed based on CD44 and CD62L (1-selectin) surface expression as memory phenotype and activation markers. The splenocytes and blood cells were stained with fluorochrome-labeled anti-CD4, anti-CD8, anti-CD20, anti-CD44, and anti-CD62L antibodies, and representative gating was used to obtain CD4+ and CD8+ T cells to analyze the expression of CD44 and CD62L. Briefly, lymphocytes were gated out based on size and complexity (FSC-a vs. SSC-a). CD20-cells were later gated on CD4+ and CD8+ markers, and the expression of CD44 and CD62L were analyzed. Although gated CD4+ and CD8+ T cells express comparable levels of CD44 and CD62L, no difference was observed in the percentage of naïve (CD44-CD62L+), TCM (CD44+CD62L+), and TE/EM (CD44+CDL−) in splenocytes and blood cells across immunized groups compared to control. Further, splenocytes and blood cells from GNP-conjugated vaccine immunized mice did not exhibit the difference in the percentage of CD20+ B cells compared to control.

This study demonstrated that peptides assembled on the DNA scaffold resulted in an efficient immunogen, producing a robust and specific immune response. Using nucleic acids as an assembly scaffold permits stoichiometrically and spatially controlled assembly of separate epitopes into a single immunogen. The rigid DNA duplex enables the segregation of individual epitopes and prevents the formation of unwanted conformational epitopes. The formulation of the immunogen is a crucial component for vaccine development. The DNA complementarity permitted the incorporation of the poly A-thiol tail, which is necessary for high-efficiency conjugation with the gold nanoparticles. The modular vaccine was assembled by chemical conjugation to gold nanoparticles (GNPs) using three conjugation strategies or co-crystallization with inulin microcrystals. The use of GNPs offers a simple formulation procedure with low toxicity and improved immunogen stability and uptake. The electrophoresis data indicates that the density of DNA coverage of the gold nanoparticle is the primary factor affecting the immunological efficiency of the GNP-immunogen construct. The thiol-gold coupling method defines the final product's density, consistent with the reported differences (Liu, et al., J. Am. Chem. Soc. (2017) 139 (28): 9471-9474). The acidic pH-based conjugation (PA) seemingly produced the highest coverage, closely followed by freezing-based (FR) conjugation. At the same time, the salt concentration-based method (SC) resulted in smearing and low mobility, suggesting low coverage and/or aggregation.

The immunogenicity of the resulting vaccines was tested and differences between each formulation was found. The inulin-based formulation had high anti-P1 antibody levels and a significantly weaker response to P2, indicating dissociation of the immunogen molecule and inefficient immunological synapse formation. P1 acts as B-cell and T-helper epitope simultaneously and thus can form immunological synapses regardless of formulation and assembly. Salt concentration-based SC formulation had a poor immune response to both peptides, suggesting poor uptake or processing by antigen-presenting cells due to aggregation or loss of antigen during dialysis. Finally, acid-(PA) and freezing-based (FR) formulations had almost identical antibody levels against both P1 and P2, indicating the stability of the formulation.

The immune responses against the full-length spike or RBD (negative control) confirmed the findings of peptide ELISA. Strong immune responses to spike with minimal immune responses to RBD were found in sera generated by acid-(PA) and freezing-based (FR) formulations and showed neutralization titers against wildtype SARS-COV-2 and delta variant (Kannan, et al., J. Autoimmun. (2021) 124:102715). The selected epitopes do not carry any of these mutations, as shown in FIG. 6, in which all mutations were mapped in the currently monitored VOCs. This finding indicates that the vaccine should be equally efficient for all circulating variants, including the omicron. Furthermore, the selected epitopes are in highly conserved or invariant regions, opening an avenue for a universal pan beta coronavirus vaccine (Xia, et al., Cell Mol. Immunol. (2020) 17 (7): 765-767; Pokhrel, et al., Sci. Rep. (2021) 11 (1): 13120). The data contrasts with the non-neutralizing and spike non-reactive antibodies generated when the peptides used are converted into an immunogen by the more traditional keyhole limpet hemocyanin (KLH) technology (Li, et al., Cell. Rep. (2021) 34 (13): 108915). This inefficacy of KLH-conjugated vaccines has been reported. The differences in the immune responses are related to the carrier-mediated folding of the epitopes into non-native conformations. On the other hand, the highly hydrophilic and rigid DNA-based carrier does not permit such interactions between the peptides and the carrier itself, thus resulting in a more native-like behavior of the peptides. The intrinsic stability of DNA, along with a strong negative charge, lends high stability to the vaccines. The gold-based formulations can be freeze-thawed 3 or more times with no aggregation and stored in a refrigerator for at least 6 months.

The developed peptide-array approach has several important features. The accessibility and affordability of solid-phase peptide synthesis of short peptide epitopes simplify the manufacturing of the vaccine (Skwarczynski, et al., Chem. Sci. (2016) 7 (2): 842-854). Programmed-complementarity DNA-based assembly of separate epitopes into a modular vaccine was used, which further improves adaptability and simplifies chemical synthesis and formulation. Most epitope vaccines utilize chemical conjugation of the epitopes to a carrier protein, such as ovalbumin or KLH, or combine the epitopes into a single fusion protein like beads on a string. Since the epitopes incorporated in such immunogens have no explicit borders, they can go through different patterns of proteolytic processing and produce a variety of new, uncharacterized, and unwanted epitopes, known as neoepitopes (Avci, et al., mSphere (2019) 4 (5): e00520-19; Romero, et al., Nat. Biomed. Eng. (2020) 4 (6): 583-584). The folding of these immunogens produces additional conformational neoepitopes, complicates storage, and results in a large batch-to-batch variability should the artificial protein have several stable structures (Koch, et al., APMIS (1996) 104(1-6):115-125). Using a rigid DNA duplex for immunogen assembly keeps the epitopes in the vaccine structurally and spatially separated, minimizing the risk of neoepitope formation. Finally, DNA has low antigenicity and does not lead to carrier-induced epitope suppression (Liu, et al., Nano Lett. (2012) 12(8):4254-4259; Jegerlehner, et al., Vaccine (2010)28(33):5503-5512). An epitope vaccine assembled on the platform can generate a precise immune response with high efficacy and no detectable shifts in lymphocyte populations, as was demonstrated by flow cytometry. As epitope vaccines in general (De Groot, et al., In: Clinical Applications of Immunomics. Falus A., editor. Springer, (2009), pp. 39-69; Oscherwitz, J., Hum. Vaccines Immunother. (2016) 12(8):2113-2116; Purcell, et al., Nat. Rev. Drug Discov. (2007)6(5):404-414; Rajcani, et al., Open Infect. Dis. J. (2018) 10(1):47-62), this modular vaccine provides protective antibodies with little or no therapeutically inefficient or harmful antibodies. This is critical if targeting some parts of the pathogen can lead to immune enhancement of the disease or adverse reactions. Additional B cell and T helper epitopes may be introduced to increase immunogenicity and the therapeutic potential. Moreover, cytotoxic T-cell epitopes may be introduced.

In conclusion, this is the first epitope vaccine made through DNA hybridization-based assembly of separate epitopes into a single molecule. Using nucleic acids is a very convenient strategy since they are non-toxic and easy to synthesize and characterize. Most importantly, they are highly programmable, allowing for reproducible and predictable assembly and long-term storage in ambient conditions. This assembly can then co-deliver the components to the antigen-presenting cells and facilitate the formation of immunological synapses. The newly developed platform also greatly simplifies the fusion of immunomodulatory ligands, such as TLR agonists, allowing for direct delivery of these ligands into the immune cells. The use of fused TLR agonists is known to significantly improve vaccine efficacy, allowing for T-cell-independent B-cell maturation (Huleatt, et al., Vaccine (2007) 25 (4): 763-775; Pihlgren, et al., Blood (2013) 121 (1): 85-94). This is especially important for the elderly and immunocompromised patients and can minimize side effects, improving the vaccine coverage in adverse event-prone populations, like individuals with autoimmune diseases (Wang, et al., Vaccines (2020) 8 (1): 128).

### Example 2

The assembly of the amyloid beta dimer from Aβ (14-23) monomers (HQKLVFFVAED (SEQ ID NO: 8)) will be made using a flexible nanoarray (FNA) template (Maity, et al., Bioconjug. Chem. (2018) 29 (6): 2755-62; Krasnoslobodtsev, et al. Biophysical J. (2015) 108:2333-2339; Maity, et al., J. Nat. Sci. (2016) 2:e187; Tong, et al., Methods (2013) 60 (2): 161-8; each incorporated herein by reference) with the following modifications. The FNA will be synthesized with DNA oligonucleotides at the ends. As before, FNA will contain dibenzocyclooctyne (DBCO) groups at selected sites within the FNA template. Peptides will be conjugated using a metal-free click chemistry, being clicked to DBCO residues through their terminal azide groups (azidolysine), to yield the FNA-dimer. Schematically, the flexible polymer will have the following structure: DNA₁-(PA)₃-DBCO-(PA)₆-DBCO-(PA)₃-DNA₂. PA are phosphoroamidite units used for the FNA synthesis. The (PA)₆ segment provides a necessary flexibility to assemble of two Aβ (14-23) monomers in the dimer (Krasnoslobodtsev, et al., Biophys J. (2015) 108 (9): 2333-9). Synthesis of the FNA oligomer will take place in an automated DNA synthesizer (Tong, et al., Methods (2013) 60 (2): 161-8).

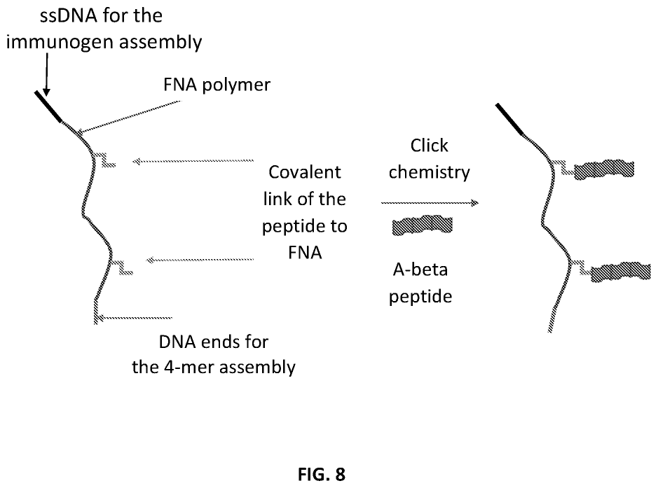

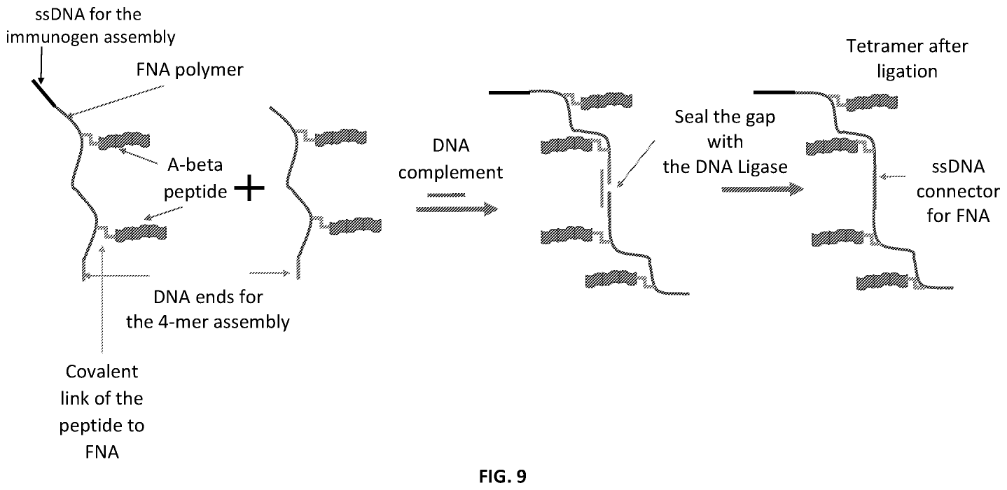

FIG. 7 provides examples of dimer assemblies. The DNA segment shown in FIG. 8 is used to assemble higher order assemblies, as an example a tetramer construct can be assembled from two dimers using a DNA complement to hybridize the DNA ends of the FNA dimers as shown in FIG. 9. The ends of the FNA dimers are ligated and the DNA complement can be removed to keep the tether flexible.

The assembly of oligomers requires the high flexibility of polymer between the peptides. FNA provides the necessary flexibility due its persistence length, as low as ˜1 nm. Single-stranded DNA has a similar persistence length (Tinland, et al., Macromolecules (1997) 30 (19): 5763-5; Roth, et al., Nano Letters (2018) 18 (11): 6703-9), so the flexibility of the FNA segment connected by ligated DNA segments after the dissociation of the DNA complement will provide the necessary flexibility to allow peptide monomers in FIG. 9 to assemble in FNA-DNA-Aβ (14-23) tetramers.

Given the high fidelity of DNA duplex formation, the annealing via DNA ends is a highly specific process allowing for the assembly of oligomers of defined sizes. One can routinely ligate sticky ends with 3-4 base pairs overhang (Sun, et al., Nanoscale Adv. (2020) 2 (3): 1318-24; Sun, et al., J. Phys. Chem. B (2021) 125 (17): 4299-307), the use of DNA on FNA as long as 5 nucleotides with the DNA complement being 10 nucleotides; the 10 bp duplex is sufficient to remain stable at typical ligation conditions (Naritsyn, et al., Mol. Biol. (1988) 22:242-6; Maity, et al., Front. Mol. Biosci. (2020) 7:69). As a result, a tetramer with monomers separated with flexible polymers is obtained.

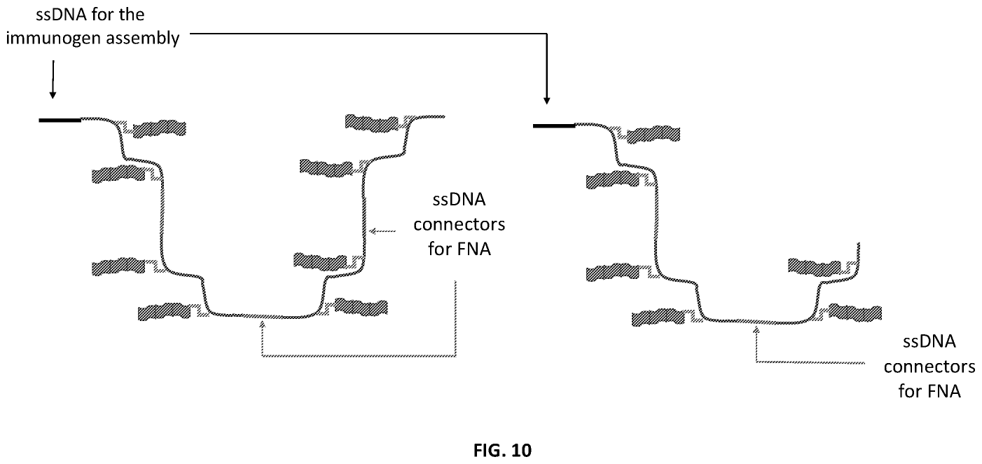

Adjusting the sequences of the DNA ends of corresponding FNA assemblies allows the formation of oligomers of any size, e.g. the ligation of two tetramers leads to an octamer, FIG. 10; a hexamer can be obtained by ligation of dimers and tetramers, FIG. 10. Again, the tether flexibility is the factor allowing for the assembly of oligomers of specific sizes. The described approach can be extended to assembly of oligomers larger than octamers and by adjusting the DNA sequences, so the immune responses to only oligomers of defined size can be achieved.

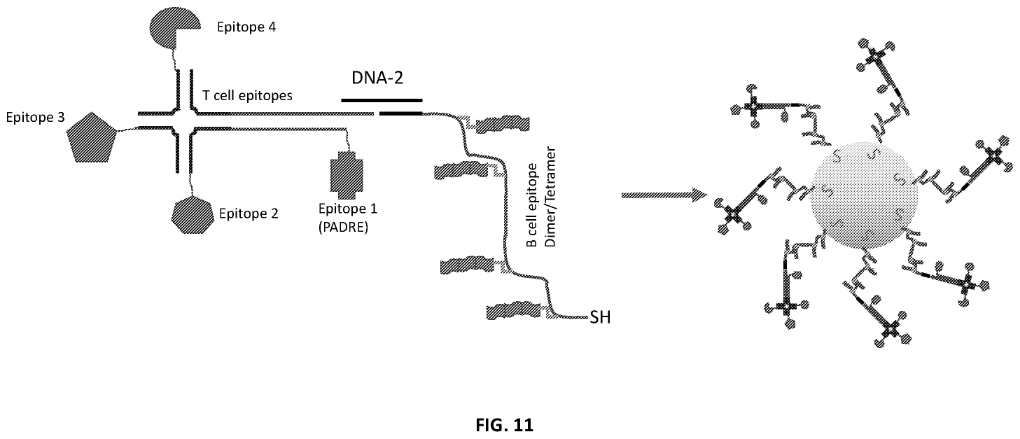

The assembly of the immunogen for the Aβ (14-23) tetramer are shown schematically in FIG. 11. The immunogen is assembled by ligation of the FNA-tetramer to the epitope assembly, which enhance the immune response as described above. The immunogen will be formulated using the gold nanoparticles by a chemical bonding of thiol groups of the FNA end.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

The invention claimed is:

1. An immunogenic construct comprising a scaffold nucleic acid molecule and at least one complementary nucleic acid molecule,
wherein said at least one complementary nucleic acid molecule is hybridized with said scaffold nucleic acid molecule, and
wherein said at least one complementary nucleic acid molecule is conjugated to a peptide antigen.

2. The immunogenic construct of claim 1, further comprising a complementary nucleic acid molecule conjugated to a T-helper cell epitope.

3. The immunogenic construct of claim 2, wherein said T-helper cell epitope comprises SEQ ID NO: 9.

4. The immunogenic construct of claim 1, wherein said immunogenic construct comprises a first complementary nucleic acid molecule conjugated to a first peptide antigen and a second complementary nucleic acid molecule conjugated to a second peptide antigen.

5. The immunogenic construct of claim 4, wherein said first peptide antigen comprises SEQ ID NO: 10 and said second peptide antigen comprises SEQ ID NO: 11.

6. The immunogenic construct of claim 1, wherein said peptide antigen comprises SEQ ID NO: 8.

7. The immunogenic construct of claim 1, further comprising a substrate for improved cellular uptake and/or biological stability conjugated to the scaffold nucleic acid molecule.

8. The immunogenic construct of claim 7, wherein the substrate is selected from the group consisting of nanoparticles, microparticles, dendrimers, nucleic acid origamis, virus-like particles, recombinant proteins, polysaccharides, metallic-based nanoparticles, carbon-based nanoparticles, polymeric nanoparticles, lipid-based nanoparticles, and ceramic nanoparticles.

9. The immunogenic construct of claim 7, wherein the substrate is gold nanoparticle.

10. The immunogenic construct of claim 1, further comprising a vaccine adjuvant.

11. The immunogenic construct of claim 1, further comprising a complementary nucleic acid molecule conjugated to a T-helper cell epitope, and
further comprising a gold nanoparticle conjugated to the scaffold nucleic acid molecule.

12. A composition comprising the immunogenic construct of claim 1 and at least one pharmaceutically acceptable carrier.

13. The composition of claim 12, further comprising a vaccine adjuvant.

14. A method for inhibiting, treating, and/or preventing a disease in a subject in need thereof, said method comprising administering to said subject an immunogenic construct of claim 1.

15. The method of claim 14, wherein said disease is a viral infection.

16. The method of claim 15, wherein said viral infection is SARS-Cov-2 and the peptide antigen is an antigen of the spike protein.

17. The method of claim 14, wherein said disease is cancer and the peptide antigen is a tumor antigen.

18. The method of claim 14, wherein said disease is Alzheimer's disease and the peptide antigen is an antigen of amyloid beta.


SEQUENCE LISTING (23 sequences)

SEQ ID NO: 1 - protein, 16 aa, synthetic construct
KGSAKFVAAWTLKAAA

SEQ ID NO: 2 - protein, 13 aa, synthetic construct
KFKEELDKYFKNH

SEQ ID NO: 3 - protein, 13 aa, synthetic construct
KTESNKKFLPFQQ

SEQ ID NO: 4 - DNA, 20 bp, synthetic construct
  source 1..20: mol_type = other DNA
tatacagcctactcactata

SEQ ID NO: 5 - DNA, 20 bp, synthetic construct
  source 1..20: mol_type = other DNA
tatactgagctagtcgtata

SEQ ID NO: 6 - DNA, 20 bp, synthetic construct
  source 1..20: mol_type = other DNA
tataccttcatccttatata

SEQ ID NO: 7 - DNA, 69 bp, synthetic construct
  source 1..69: mol_type = other DNA
aaaaaaaaatatagtgagtaggctgtatatatacgactagctcagtatatatataaggatgaaggtata

SEQ ID NO: 8 - protein, 11 aa, synthetic construct
HQKLVFFVAED

SEQ ID NO: 9 - protein, 15 aa, synthetic construct
GSAKFVAAWTLKAAA

SEQ ID NO: 10 - protein, 12 aa, synthetic construct
FKEELDKYFKNH

SEQ ID NO: 11 - protein, 12 aa, synthetic construct
TESNKKFLPFQQ

SEQ ID NO: 12 - DNA, 12 bp, synthetic construct
  source 1..12: mol_type = other DNA
cgtaagtagtca

SEQ ID NO: 13 - DNA, 12 bp, synthetic construct
  source 1..12: mol_type = other DNA
tgactgcttact

SEQ ID NO: 14 - DNA, 68 bp, synthetic construct
  source 1..68: mol_type = other DNA
ccttcatccttactgagctagtcgaacgtcaggaacgtcatggaccttgcgacagacagcctactcac

SEQ ID NO: 15 - DNA, 12 bp, synthetic construct
  source 1..12: mol_type = other DNA
taaggatgaagg

SEQ ID NO: 16 - DNA, 12 bp, synthetic construct
  source 1..12: mol_type = other DNA
cgactagctcag

SEQ ID NO: 17 - DNA, 20 bp, synthetic construct
  source 1..20: mol_type = other DNA
tccatgacgttcctgacgtt

SEQ ID NO: 18 - DNA, 12 bp, synthetic construct
  source 1..12: mol_type = other DNA
tctgtcgcaagg

SEQ ID NO: 19 - DNA, 12 bp, synthetic construct
  source 1..12: mol_type = other DNA
gtgagtaggctg

SEQ ID NO: 20 - protein, 33 aa, synthetic construct
FSPDGKPCTPPALNCYWPLNDYGFYTTTGIGYQ

SEQ ID NO: 21 - protein, 17 aa, synthetic construct
FSPDGKPATPPALNAYW

SEQ ID NO: 22 - skipped

SEQ ID NO: 23 - DNA, 60 bp, synthetic construct
  source 1..60: mol_type = other DNA
tatagtgagtaggctgtatatatacgactagctcagtatatatataaggatgaaggtata